SEQUENCE LISTING (33 sequences)

SEQ ID NO: 1 - protein, 248 aa, Homo sapiens
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 2 - protein, 228 aa, Homo sapiens
ETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 3 - protein, 141 aa, Homo sapiens
AASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 4 - protein, 115 aa, Homo sapiens
VGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 5 - protein, 148 aa, Homo sapiens
PDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 6 - protein, 381 aa, Artificial Sequence
  Recombinantly-produced fusion protein
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEINITFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 7 - protein, 383 aa, Artificial Sequence
  Recombinantly-produced fusion protein
AKTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 8 - protein, 352 aa, Artificial Sequence
  Recombinantly-produced fusion protein
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGATSKQVGNKFFLTNGEEVITFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 9 - protein, 383 aa, Artificial Sequence
  Recombinantly-produced fusion protein
AKTEPKSSDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 10 - protein, 10 aa, Homo sapiens
NEGEPNNAGS

SEQ ID NO: 11 - protein, 15 aa, Homo sapiens
LLLKNGQWNDVPCST

SEQ ID NO: 12 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 13 - protein, 278 aa, Homo sapiens [sequence omitted]

SEQ ID NO: 14 - protein, 375 aa, Homo sapiens
MLLFLLSALVLLTQPLGYLEAEMKTYSHRTMPSACTLVMCSSVESGLPGRDGRDGREGPRGEKGDPGLPGAAGQAGMPGQAGPVGPKGDNGSVGEPGPKGDTGPSGPPGPPGVPGPAGREGALGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 15 - protein, 277 aa, Homo sapiens
MNGFASLLRRNQFILLVLFLLQIQSLGLDIDSRPTAEVCATHTISPGPKGDDGEKGDPGEEGKHGKVGRMGPKGIKGELGDMGDQGNIGKTGPIGKKGDKGEKGLLGIPGEKGKAGTVCDCGRYRKFVGQLDISIARLKTSMKFVKNVIAGIRETEEKFYYIVQEEKNYRESLTHCRIRGGMLAMPKDEAANTLIADYVAKSGFFRVFIGVNDLEREGQYMFTDNTPLQNYSNWNEGEPSDPYGHEDCVEMLSSGRWNDTECHLTMYFVCEFIKKKK

SEQ ID NO: 16 - protein, 742 aa, Homo sapiens
MKDDFAEEEEVQSFGYKRFGIQEGTQCTKCKNNWALKFSIILLYILCALLTITVAILGYKVVEKMDNVTGGMETSRQTYDDKLTAVESDLKKLGDQTGKKAISTNSELSTFRSDILDLRQQLREITEKTSKNKDTLEKLQASGDALVDRQSQLKETLENNSFLITTVNKTLQAYNGYVTNLQQDTSVLQGNLQNQMYSHNVVIMNLNNLNLTQVQQRNLITNLQRSVDDTSQAIQRIKNDFQNLQQVFLQAKKDTDWLKEKVQSLQTLAANNSALAKANNDTLEDMNSQLNSFTGQMENITTISQANEQNLKDLQDLHKDAENRTAIKFNQLEERFQLFETDIVNIISNISYTAHHLRTLTSNLNEVRTTCTDTLTKHTDDLTSLNNTLANIRLDSVSLRMQQDLMRSRLDTEVANLSVIMEEMKLVDSKHGQLIKNFTILQGPPGPRGPRGDRGSQGPPGPTGNKGQKGEKGEPGPPGPAGERGPIGPAGPPGERGGKGSKGSQGPKGSRGSPGKPGPQGPSGDPGPPGPPGKEGLPGPQGPPGFQGLQGTVGEPGVPGPRGLPGLPGVPGMPGPKGPPGPPGPSGAVVPLALQNEPTPAPEDNGCPPHWKNFTDKCYYFSVEKEIFEDAKLFCEDKSSHLVFINTREEQQWIKKQMVGRESHWIGLTDSERENEWKWLDGTSPDYKNWKAGQPDNWGHGHGPGEDCAGLIYAGQWNDFQCEDVNNFICEKDRETVLSSAL

SEQ ID NO: 17 - protein, 321 aa, Bos taurus
MLPLPLSILLLLTQSQSFLGEEMDVYSEKTLTDPCTVVVCAPPADSLRGHDGRDGKEGPQGEKGDPGPPGMPGPAGREGPSGRQGSMGPPGTPGPKGEPGPEGGVGAPGMPGSPGPTGLKGERGTPGPGGAIGPQGPSGAMGPPGLKGDRGDPGEKGAKGETSVLEVDTLRQRMRNLEGEVQRLQNIVTQYRKAVLFPDGQAVGEKIFKTAGAVKSYSDAEQLCREAKGQLASPRSSAENEAVTQLVRAKNKHAYLSMNDISKEGKFTYPTGGSLDYSNWAPGEPNNRAKDEGPENCLEIYSDGNWNDIECREERLVICEF

SEQ ID NO: 18 - protein, 371 aa, Bos taurus
MLLLPLSVLLLLTQPWRSLGAEMKIYSQKTLANGCTLVVCRPPEGGLPGRDGQDGREGPQGEKGDPGSPGPAGRAGRPGPAGPIGPKGDNGSAGEPGPKGDTGPPGPPGMPGPAGREGPSGKQGSMGPPGTPGPKGDTGPKGGMGAPGMQGSPGPAGLKGERGAPGELGAPGSAGVAGPAGAIGPQGPSGARGPPGLKGDRGDPGERGAKGESGLADVNALKQRVTILEGQLQRLQNAFSRYKKAVLFPDGQAVGKKIFKTAGAVKSYSDAQQLCREAKGQLASPRSAAENEAVAQLVRAKNNDAFLSMNDISTEGKFTYPTGESLVYSNWASGEPNNNNAGQPENCVQIYREGKWNDVPCSEPLLVICEF

SEQ ID NO: 19 - protein, 271 aa, Homo sapiens
MRGNLALVGVLISLAFLSLLPSGHPQPAGDDACSVQILVPGLKGDAGEKGDKGAPGRPGRVGPTGEKGDMGDKGQKGSVGRHGKIGPIGSKGEKGDSGDIGPPGPNGEPGLPCECSQLRKAIGEMDNQVSQLTSELKFIKNAVAGVRETESKIYLLVKEEKRYADAQLSCQGRGGTLSMPKDEAANGLMAAYLAQAGLARVFIGINDLEKEGAFVYSDHSPMRTFNKWRSGEPNNAYDEEDCVEMVASGGWNDVACHTTMYFMCEFDKENM

SEQ ID NO: 20 - protein, 371 aa, Bos taurus
MLLLPLSVLLLLTQPWRSLGAEMTTFSQKILANACTLVMCSPLESGLPGHDGQDGRECPHGEKGDPGSPGPAGRAGRPGWVGPIGPKGDNGFVGEPGPKGDTGPRGPPGMPGPAGREGPSGKQGSMGPPGTPGPKGETGPKGGVGAPGIQGFPGPSGLKGEKGAPGETGAPGRAGVTGPSGAIGPQGPSGARGPPGLKGDRGDPGETGAKGESGLAEVNALKQRVTILDGHLRRFQNAFSQYKKAVLFPDGQAVGEKIFKTAGAVKSYSDAEQLCREAKGQLASPRSSAENEAVTQMVRAQEKNAYLSMNDISTEGRFTYPTGEILVYSNWADGEPNNSDEGQPENCVEIFPDGKWNDVPCSKQLLVICEF

SEQ ID NO: 21 - protein, 326 aa, Homo sapiens
MELSGATMARGLAVLLVLFLHIKNLPAQAADTCPEVKVVGLEGSDKLTILRGCPGLPGAPGPKGEAGVIGERGERGLPGAPGKAGPVGPKGDRGEKGMRGEKGDAGQSQSCATGPRNCKDLLDRGYFLSGWHTIYLPDCRPLTVLCDMDTDGGGWTVFQRRMDGSVDFYRDWAAYKQGFGSQLGEFWLGNDNIHALTAQGSSELRVDLVDFEGNHQFAKYKSFKVADEAEKYKLVLGAFVGGSAGNSLTGHNNNFFSTKDQDNDVSSSNCAEKFQGAWWYADCHASNLNGLYLMGPHESYANGINWSAAKGYKYSYKVSEMKVRPA

SEQ ID NO: 22 - protein, 313 aa, Homo sapiens
MELDRAVGVLGAATLLLSFLGMAWALQAADTCPEVKMVGLEGSDKLTILRGCPGLPGAPGPKGEAGTNGKRGERGPPGPPGKAGPPGPNGAPGEPQPCLTGPRTCKDLLDRGHFLSGWHTIYLPDCRPLTVLCDMDTDGGGWTVFQRRVDGSVDFYRDWATYKQGFGSRLGEFWLGNDNIHALTAQGTSELRVDLVDFEDNYQFAKYRSFKVADEAEKYNLVLGAFVEGSAGDSLTFHNNQSFSTKDQDNDLNTGNCAVMFQGAWWYKNCHVSNLNGRYLRGTHGSFANGINWKSGKGYNYSYKVSEMKVRPA

SEQ ID NO: 23 - protein, 299 aa, Homo sapiens
MDLLWILPSLWLLLLGGPACLKTQEHPSCPGPRELEASKVVLLPSCPGAPGSPGEKGAPGPQGPPGPPGKMGPKGEPGDPVNLLRCQEGPRNCRELLSQGATLSGWYHLCLPEGRALPVFCDMDTEGGGWLVFQRRQDGSVDFFRSWSSYRAGFGNQESEFWLGNENLHQLTLQGNWELRVELEDFNGNRTFAHYATFRLLGEVDHYQLALGKFSEGTAGDSLSLHSGRPFTTYDADHDSSNSNCAVIVHGAWWYASCYRSNLNGRYAVSEAAAHKYGIDWASGRGVGHPYRRVRMMLR

SEQ ID NO: 24 - protein, 786 aa, Homo sapiens
MTSIFHFAIIFMLILQIRIQLSEESEFLVDRSKNGLIHVPKDLSQKTTILNISQNYISELWTSDILSLSKLRILIISHNRIQYLDISVFKFNQELEYLDLSHNKLVKISCHPTVNLKHLDLSFNAFDALPICKEFGNMSQLKFLGLSTTHLEKSSVLPIAHLNISKVLLVLGETYGEKEDPEGLQDFNTESLHIVFPTNKEFHFILDVSVKTVANLELSNIKCVLEDNKCSYFLSILAKLQTNPKLSNLTLNNIETTWNSFIRILQLVWHTTVWYFSISNVKLQGQLDFRDFDYSGTSLKALSIHQVVSDVFGFPQSYIYEIFSNMNIKNFTVSGTRMVHMLCPSKISPFLHLDFSNNLLTDTVFENCGHLTELETLILQMNQLKELSKIAEMTTQMKSLQQLDISQNSVSYDEKKGDCSWTKSLLSLNMSSNILTDTIFRCLPPRIKVLDLHSNKIKSIPKQVVKLEALQELNVAFNSLTDLPGCGSFSSLSVLIIDHNSVSHPSADFFQSCQKMRSIKAGDNPFQCTCELGEFVKNIDQVSSEVLEGWPDSYKCDYPESYRGTLLKDFHMSELSCNITLLIVTIVATMLVLAVTVTSLCSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 25 - protein, 784 aa, Homo sapiens
MPHTLWMVWVLGVIISLSKEESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRTALVSGMCCALFLLILLTGVLCHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 26 - protein, 904 aa, Homo sapiens
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 27 - protein, 839 aa, Homo sapiens
MMSASRLAGTLIPAMAFLSCVRPESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 28 - protein, 858 aa, Homo sapiens
MGDHLDLLLGVVLMAGPVFGIPSCSFDGRIAFYRFCNLTQVPQVLNTTERLLLSFNYIRTVTASSFPFLEQLQLLELGSQYTPLTIDKEAFRNLPNLRILDLGSSKIYFLHPDAFQGLFHLFELRLYFCGLSDAVLKDGYFRNLKALTRLDLSKNQIRSLYLHPSFGKLNSLKSIDFSSNQIFLVCEHELEPLQGKTLSFFSLAANSLYSRVSVDWGKCMNPFRNMVLEILDVSGNGWTVDITGNFSNAISKSQAFSLILAHHIMGAGFGFHNIKDPDQNTFAGLARSSVRHLDLSHGFVFSLNSRVFETLKDLKVLNLAYNKINKIADEAFYGLDNLQVLNLSYNLLGELYSSNFYGLPKVAYIDLQKNHIAIIQDQTFKFLEKLQTLDLRDNALTTIHFIPSIPDIFLSGNKLVTLPKINLTANLIHLSENRLENLDILYFLLRVPHLQILILNQNRFSSCSGDQTPSENPSLEQLFLGENMLQLAWETELCWDVFEGLSHLQVLYLNHNYLNSLPPGVFSHLTALRGLSLNSNRLTVLSHNDLPANLEILDISRNQLLAPNPDVFVSLSVLDITHNKFICECELSTFINWLNHTNVTIAGPPADIYCVYPDSFSGVSLFSLSTEGCDEEEVLKSLKFSLFIVCTVTLTLFLMTILTVTKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDLQDVGWFLHKLSQQILKKEKEKKKDNNIPLQTVATIS

SEQ ID NO: 29 - protein, 796 aa, Homo sapiens
MTKDKEPIVKSFHFVCLMIIIVGTRIQFSDGNEFAVDKSKRGLIHVPKDLPLKTKVLDMSQNYIAELQVSDMSFLSELTVLRLSHNRIQLLDLSVFKFNQDLEYLDLSHNQLQKISCHPIVSFRHLDLSFNDFKALPICKEFGNLSQLNFLGLSAMKLQKLDLLPIAHLHLSYILLDLRNYYIKENETESLQILNAKTLHLVFHPTSLFAIQVNISVNTLGCLQLTNIKLNDDNCQVFIKFLSELTRGSTLLNFTLNHIETTWKCLVRVFQFLWPKPVEYLNIYNLTIIESIREEDFTYSKTTLKALTIEHITNQVFLFSQTALYTVFSEMNIMMLTISDTPFIHMLCPHAPSTFKFLNFTQNVFTDSIFEKCSTLVKLETLILQKNGLKDLFKVGLMTKDMPSLEILDVSWNSLESGRHKENCTWVESIVVLNLSSNMLTDSVFRCLPPRIKVLDLHSNKIKSVPKQVVKLEALQELNVAFNSLTDLPGCGSFSSLSVLIIDHNSVSHPSADFFQSCQKMRSIKAGDNPFQCTCELREFVKNIDQVSSEVLEGWPDSYKCDYPESYRGSPLKDFHMSELSCNITLLIVTIGATMLVLAVTVTSLCIYLDLPWYLRMVCQWTQTRRRARNIPLEELQRNLQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAFNMKLTLVTENNDVKS

SEQ ID NO: 30 - protein, 1049 aa, Homo sapiens
MVFPMWTLKRQILILFNIILISKLLGARWFPKTLPCDVTLDVPKNHVIVDCTDKHLTEIPGGIPTNTTNLTLTINHIPDISPASFHRLDHLVEIDFRCNCVPIPLGSKNNMCIKRLQIKPRSFSGLTYLKSLYLDGNQLLEIPQGLPPSLQLLSLEANNIFSIRKENLTELANIEILYLGQNCYYRNPCYVSYSIEKDAFLNLTKLKVLSLKDNNVTAVPTVLPSTLTELYLYNNMIAKIQEDDFNNLNQLQILDLSGNCPRCYNAPFPCAPCKNNSPLQIPVNAFDALTELKVLRLHSNSLQHVPPRWFKNINKLQELDLSQNFLAKEIGDAKFLHFLPSLIQLDLSFNFELQVYRASMNLSQAFSSLKSLKILRIRGYVFKELKSFNLSPLHNLQNLEVLDLGTNFIKIANLSMFKQFKRLKVIDLSVNKISPSGDSSEVGFCSNARTSVESYEPQVLEQLHYFRYDKYARSCRFKNKEASFMSVNESCYKYGQTLDLSKNSIFFVKSSDFQHLSFLKCLNLSGNLISQTLNGSEFQPLAELRYLDFSNNRLDLLHSTAFEELHKLEVLDISSNSHYFQSEGITHMLNFTKNLKVLQKLMMNDNDISSSTSRTMESESLRTLEFRGNHLDVLWREGDNRYLQLFKNLLKLEELDISKNSLSFLPSGVFDGMPPNLKNLSLAKNGLKSFSWKKLQCLKNLETLDLSHNQLTTVPERLSNCSRSLKNLILKNNQIRSLTKYFLQDAFQLRYLDLSSNKIQMIQKTSFPENVLNNLKMLLLHHNRFLCTCDAVWFVWWVNHTEVTIPYLATDVTCVGPGAHKGQSVISLDLYTCELDLTNLILFSLSISVSLFLMVMMTASHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 31 - protein, 1041 aa, Homo sapiens
MENMFLQSSMLTCIFLLISGSCELCAEENFSRSYPCDEKKQNDSVIAECSNRRLQEVPQTVGKYVTELDLSDNFITHITNESFQGLQNLTKINLNHNPNVQHQNGNPGIQSNGLNITDGAFLNLKNLRELLLEDNQLPQIPSGLPESLTELSLIQNNIYNITKEGISRLINLKNLYLAWNCYFNKVCEKTNIEDGVFETLTNLELLSLSFNSLSHVPPKLPSSLRKLFLSNTQIKYISEEDFKGLINLTLLDLSGNCPRCFNAPFPCVPCDGGASINIDRFAFQNLTQLRYLNLSSTSLRKINAAWFKNMPHLKVLDLEFNYLVGEIASGAFLTMLPRLEILDLSFNYIKGSYPQHINISRNFSKLLSLRALHLRGYVFQELREDDFQPLMQLPNLSTINLGINFIKQIDFKLFQNFSNLEIIYLSENRISPLVKDTRQSYANSSSFQRHIRKRRSTDFEFDPHSNFYHFTRPLIKPQCAAYGKALDLSLNSIFFIGPNQFENLPDIACLNLSANSNAQVLSGTEFSAIPHVKYLDLTNNRLDFDNASALTELSDLEVLDLSYNSHYFRIAGVTHHLEFIQNFTNLKVLNLSHNNIYTLTDKYNLESKSLVELVFSGNRLDILWNDDDNRYISIFKGLKNLTRLDLSLNRLKHIPNEAFLNLPASLTELHINDNMLKFFNWTLLQQFPRLELLDLRGNKLLFLTDSLSDFTSSLRTLLLSHNRISHLPSGFLSEVSSLKHLDLSSNLLKTINKSALETKTTTKLSMLELHGNPFECTCDIGDFRRWMDEHLNVKIPRLVDVICASPGDQRGKSIVSLELTTCVSDVTAVILFFFTFFITTMVMLAALAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 32 - protein, 1032 aa, Homo sapiens
MGFCRSALHPLSLLVQAIMLAMTLALGTLPAFLPCELQPHGLVNCNWLFLKSVPHFSMAAPRGNVTSLSLSSNRIHHLHDSDFAHLPSLRHLNLKWNCPPVGLSPMHFPCHMTIEPSTFLAVPTLEELNLSYNNIMTVPALPKSLISLSLSHTNILMLDSASLAGLHALRFLFMDGNCYYKNPCRQALEVAPGALLGLGNLTHLSLKYNNLTVVPRNLPSSLEYLLLSYNRIVKLAPEDLANLTALRVLDVGGNCRRCDHAPNPCMECPRHFPQLHPDTFSHLSRLEGLVLKDSSLSWLNASWFRGLGNLRVLDLSENFLYKCITKTKAFQGLTQLRKLNLSFNYQKRVSFAHLSLAPSFGSLVALKELDMHGIFFRSLDETTLRPLARLPMLQTLRLQMNFINQAQLGIFRAFPGLRYVDLSDNRISGASELTATMGEADGGEKVWLQPGDLAPAPVDTPSSEDFRPNCSTLNFTLDLSRNNLVTVQPEMFAQLSHLQCLRLSHNCISQAVNGSQFLPLTGLQVLDLSHNKLDLYHEHSFTELPRLEALDLSYNSQPFGMQGVGHNFSFVAHLRTLRHLSLAHNNIHSQVSQQLCSTSLRALDFSGNALGHMWAEGDLYLHFFQGLSGLIWLDLSQNRLHTLLPQTLRNLPKSLQVLRLRDNYLAFFKWWSLHFLPKLEVLDLAGNQLKALTNGSLPAGTRLRRLDVSCNSISFVAPGFFSKAKELRELNLSANALKTVDHSWFGPLASALQILDVSANPLHCACGAAFMDFLLEVQAAVPGLPSRVKCGSPGQLQGLSIFAQDLRLCLDEALSWDCFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 33 - protein, 811 aa, Homo sapiens
MRLIRNIYIFCSIVMTAEGDAPELPEERELMTNCSNMSLRKVPADLTPATTTLDLSYNLLFQLQSSDFHSVSKLRVLILCHNRIQQLDLKTFEFNKELRYLDLSNNRLKSVTWYLLAGLRYLDLSFNDFDTMPICEEAGNMSHLEILGLSGAKIQKSDFQKIAHLHLNTVFLGFRTLPHYEEGSLPILNTTKLHIVLPMDTNFWVLLRDGIKTSKILEMTNIDGKSQFVSYEMQRNLSLENAKTSVLLLNKVDLLWDDLFLILQFVWHTSVEHFQIRNVTFGGKAYLDHNSFDYSNTVMRTIKLEHVHFRVFYIQQDKIYLLLTKMDIENLTISNAQMPHMLFPNYPTKFQYLNFANNILTDELFKRTIQLPHLKTLILNGNKLETLSLVSCFANNTPLEHLDLSQNLLQHKNDENCSWPETVVNMNLSYNKLSDSVFRCLPKSIQILDLNNNQIQTVPKETIHLMALRELNIAFNFLTDLPGCSHFSRLSVLNIEMNFILSPSLDFVQSCQEVKTLNAGRNPFRCTCELKNFIQLETYSEVMMVGWSDSYTCEYPLNLRGTRLKDVHLHELSCNTALLIVTIVVIMLVLGLAVAFCCLHFDLPWYLRMLGQCTQTWHRVRKTTQEQLKRNVRFHAFISYSEHDSLWVKNELIPNLEKEDGSILICLYESYFDPGKSISENIVSFIEKSYKSIFVLSPNFVQNEWCHYEFYFAHHNLFHENSDHIILILLEPIPFYCIPTRYHKLKALLEKKAYLEWPKDRRKCGLFWANLRAAINVNVLATREMYELQTFTELNEESRGSTISLMRTDCL

(19) United States
(12) Patent Application Publication
(10) Pub. No.: US 2023/0416319 A1
(43) Pub. Date: Dec. 28, 2023

(54) COMPOSITIONS, DEVICES AND METHODS FOR IDENTIFYING, TREATING AND PREVENTING INFECTIOUS DISEASE USING MICROBE-TARGETING MOLECULES

(71) Applicant: MIRAKI INNOVATION THINK TANK LLC, Cambridge, MA (US)

(72) Inventors: Keith CRAWFORD, Westwood, MA (US); Christopher VELIS, Lexington, MA (US); Goossen Jan Bernard BOER, Brookline, MA (US); George A. DOWNEY, Arlington, MA (US)

(73) Assignee: MIRAKI INNOVATION THINK TANK LLC, Cambridge, MA (US)

(21) Appl. No.: 18/039,063

(22) PCT Filed: Sep. 30, 2021
(86) PCT No.: PCT/US2021/052934

§ 371 Date: May 26, 2023

Related U.S. Application Data
(60) Provisional application No. 63/086,435, filed on Oct. 1, 2020.

(51) Int. Cl. C07K 14/47; C07K 14/705; G01N 33/569

(52) U.S. Cl. CPC C07K 14/4726; C07K 14/705; G01N 33/569

(57) ABSTRACT

Compositions, devices and methods for capturing, detecting and identifying one or more microbes or microbe components and/or treating infectious disease using microbe-targeting molecules (MTMs) are provided.

### SEQUENCE LISTING

A sequence listing in electronic (ASCII text file) format is filed with this application and incorporated herein by reference. The name of the ASCII text file is “2021_2009A_ST25.txt”; the file was created on Sep. 30, 2021; the size of the file is 132 KB.

### FIELD OF THE INVENTION

The technology described herein relates to compositions, devices and methods for capturing, detecting and/or identifying one or more microbes or microbe components and/or treating or preventing infectious disease.

### BACKGROUND

Sepsis is a life-threatening condition that results from microbial infections (e.g., bacterial, viral, parasitic, or fungal) and the body's associated response causing damage to tissues. Sepsis is a major cause of death in American intensive care units. While microbes can directly damage tissues, resulting inflammatory responses can cause further damage and lead to septic shock and death. Early detection of infection and accurate identification of the infecting microbes are keys to successful treatment as different microbes are most susceptible to different treatments. Patients in septic shock should be treated as soon as possible to derive optimal benefit from antimicrobial therapies. Further, means and methods for prevention, early intervention and treatment of infectious disease are needed. The present invention is directed to these and other important goals.

### BRIEF SUMMARY

The present invention is generally directed to compositions, devices and methods for capturing, detecting and/or identifying one or more microbes or microbe components and/or treating or preventing infectious disease in a subject (e.g., a human or an animal subject) using microbe-targeting molecules (MTMs) and engineered MTMs as defined herein.

### MTMs

As summarized above, the invention is directed, in part, to “microbe-targeting molecules” (MTMs) and engineered MTMs that have the shared characteristic of binding to one or more microbe-associated molecular patterns (MAMPs). MTMs distinguish and bind microbes and microbial components from a sample based on the identity of the MAMP produced by the microbe, rather than the identity of microbe itself. While some MAMPs are produced by only a single species of microbe, other MAMPs are shared across species. Thus, while some MTMs of the invention bind to only MAMPs of a particular species of microbe, other MTMs of the invention can bind to MAMPs produced by all members of a particular class, order, family, genus or sub-genus of microbe.

As used herein, “MTM” and “engineered MTM” refers to any of the molecules described herein (or described in patents or patent application incorporated by reference) that can bind to a microbe or microbe component. Unless the context indicates otherwise, the term “MTM” is used to describe all MTMs of the invention, both naturally-occurring and engineered forms of these constructs.

MTMs can be used to contact, and optionally isolate, microbes and microbial components from a sample or in the environment based on the identity of the MAMP produced by the microbe, rather than the identity of microbe itself. While some MAMPs are produced by only a single species of microbe, other MAMPs are shared across species.

Given that the MTMs of the invention are defined based on their binding activity, it will be apparent that both naturally-occurring and engineered MTMs will comprise at least one microbe-binding domain, i.e. a domain that recognizes and binds to one or more MAMPs (including, at least two, at least three, at least four, at least five, or more) as described herein. A microbe-binding domain can be a naturally-occurring or a synthetic molecule. In some aspects, a microbe-binding domain can be a recombinant molecule. In addition to the microbe-binding domain, the MTMs of the invention will typically have one or more additional domains that may include, but are not limited to, an oligomerization domain, a signal domain, an anchor domain, a collagen-like domain, a fibrinogen-like domain, an immunoglobulin domain, and an immunoglobulin-like domain.

As non-limiting examples of the MTMs of the invention, three broad categories of MTMs are encompassed within the invention, namely: (i) collectin-based MTMs, (ii) ficolin-based MTMs, and (iii) toll-like receptor-based MTMs.

Thus, and in a first embodiment, the present invention is directed to compositions comprising collectin-based MTMs. The collectin-based MTMs are either naturally-occurring collectin proteins or engineered MTM fusion proteins that comprise at least one collectin microbe-binding domain and at least one additional domain.

The naturally-occurring collectin protein may be any one of (i) mannose-binding lectin (MBL), (ii) surfactant protein A (SP-A), (iii) surfactant protein D (SP-D), (iv) collectin liver 1 (CL-L1), (v) collectin placenta 1 (CL-P1), (vi) conglutinin collectin of 43 kDa (CL-43), (vii) collectin of 46 kDa (CL-46), (viii) collectin kidney 1 (CL-K1), (ix) conglutinin, and (x) a sequence variant having at least 85% sequence identity to any one of (i)-(ix).

In one aspect of this embodiment, the collectin is (i) a naturally-occurring MBL, (ii) a truncated form of naturally-occurring MBL, (iii) an engineered form of MBL, or (iv) a sequence variant having at least 85% sequence identity to any one of (i), (ii) or (iii).

In a specific aspect of this embodiment, the collectin is a naturally-occurring MBL as set forth in SEQ ID NO:1 or a sequence variant having at least 85% sequence identity with SEQ ID NO:1 that retains the activity of the native protein.

In another specific aspect of this embodiment, the collectin is a truncated form of naturally-occurring MBL as set forth in any one of SEQ ID NOs:2-5 or a sequence variant having at least 85% sequence identity with any one of SEQ ID NOs:2-5 that retains the activity of the native protein.

The collectin microbe-binding domain of the collectin-based engineered MTMs comprises a carbohydrate recognition domain (CRD) of a collectin. The collectin may be any one of (i) mannose-binding lectin (MBL), (ii) surfactant protein A (SP-A), (iii) surfactant protein D (SP-D), (iv) collectin liver 1 (CL-L1), (v) collectin placenta 1 (CL-P1), (vi) conglutinin collectin of 43 kDa (CL-43), (vii) collectin of 46 kDa (CL-46), (viii) collectin kidney 1 (CL-K1), (ix) conglutinin, and (x) a sequence variant having at least 85% sequence identity to any one of (i)-(ix).

The at least one additional domain of the collectin-based engineered MTMs may be one or more of (xi) a collectin cysteine-rich domain, (xii) a collectin collagen-like domain, (xiii) a collectin coiled-coil neck domain, (xiv) a ficolin short N-terminal domain, (xv) a ficolin collagen-like domain, (xvi) a Toll-like receptor (TLR) transmembrane helix, (xvii) a TLR C-terminal cytoplasmic signaling domain, (xviii) an oligomerization domain, (xix) a signal domain, (xx) an anchor domain, (xxi) a collagen-like domain, (xxii) a fibrinogen-like domain, (xxiii) an immunoglobulin domain, (xxiv) an immunoglobulin-like domain, and (xxv) a sequence variant having at least 85% sequence identity to any one of (xi)-(xxiv).

In certain aspects of this embodiment, the at least one additional domain is an immunoglobulin domain. For example, the immunoglobulin domain may comprise the amino acid sequence of SEQ ID NO:12 or a sequence variant thereof having at least 85% sequence identity to SEQ ID NO:12.

In certain aspects of this embodiment, the collectin microbe-binding domain comprises the CRD of MBL or a sequence variant thereof having at least 85% sequence identity to the CRD of MBL. For example, the CRD of MBL may comprise the amino acid sequence of any one of SEQ ID NOs:1, 2, 3, 4, and 5 or a sequence variant thereof having at least 85% sequence identity to any one of SEQ ID NOs:1, 2, 3, 4, and 5.

In certain aspects of this embodiment, the MTM comprises the CRD of MBL or a sequence variant thereof having at least 85% sequence identity to the CRD of MBL and an immunoglobulin domain. For example, the CRD of MBL may comprise the amino acid sequence of any one of SEQ ID NOs:1, 2, 3, 4, and 5 or a sequence variant thereof having at least 85% sequence identity to any one of SEQ ID NOs:1, 2, 3, 4, and 5, and an immunoglobulin domain comprising the amino acid sequence of SEQ ID NO12: or a sequence variant thereof having at least 85% sequence identity to SEQ ID NO:12.

In certain aspects of this embodiment, the compositions comprise at least one collectin-based engineered MTM, wherein the collectin-based engineered MTM is an FcMBL of SEQ ID NO:6, 7, 8 or 9, or a sequence variant thereof having at least 85% sequence identity to SEQ ID NO:6, 7, 8 or 9. FcMBL MTMs comprise a mannose-binding ligand (MBL) linked to the Fc domain of human IgG (Fc).

In certain other aspects of this embodiment, the compositions comprise at least two collectin-based engineered MTMs, wherein the two MTMs are selected from SEQ ID NO:6, 7, 8 or 9, or sequence variants thereof having at least 85% sequence identity to SEQ ID NO:6, 7, 8 or 9.

In a second embodiment, the invention is directed to compositions comprising ficolin-based MTMs. The ficolin-based MTMs are either naturally-occurring ficolin proteins or engineered MTM fusion proteins that comprise at least one ficolin microbe-binding domain and at least one additional domain.

The naturally-occurring ficolin protein may be any one of (i) ficolin 1, (ii) ficolin 2, (iii) ficolin 3, and (iv) a sequence variant having at least 85% sequence identity to any one of (i)-(iii).

The ficolin microbe-binding domain of the ficolin-based engineered MTMs may comprise the fibrinogen-like domain of a ficolin. The ficolin may be any one of (i) ficolin 1, (ii) ficolin 2, (iii) ficolin 3, and (iv) a sequence variant having at least 85% sequence identity to any one of (i)-(iii).

The at least one additional domain of the ficolin-based engineered MTMs may be one or more of (v) a ficolin short N-terminal domain, (vi) a ficolin collagen-like domain, (vii) a collectin cysteine-rich domain, (viii) a collectin collagen-like domain, (ix) a collectin coiled-coil neck domain, (x) a TLR transmembrane helix, (xi) a TLR C-terminal cytoplasmic signaling domain, (xii) an oligomerization domain, (xiii) a signal domain, (xiv) an anchor domain, (xv) a collagen-like domain, (xvi) a fibrinogen-like domain, (xvii) an immunoglobulin domain, (xviii) an immunoglobulin-like domain, and (xix) a sequence variant having at least 85% sequence identity to any one of (v)-(xviii).

In certain aspects of this embodiment, the at least one additional domain is an immunoglobulin domain. For example, the immunoglobulin domain may comprise the amino acid sequence of SEQ ID NO:12 or a sequence variant thereof having at least 85% sequence identity to SEQ ID NO:12.

In certain aspects of this embodiment, the ficolin-based engineered MTMs comprise a ficolin microbe-binding domain comprising the fibrinogen-like domain of any one of SEQ ID NOs: 21, 22 and 23 or a sequence variant thereof having at least 85% sequence identity to any one of SEQ ID NOs:21, 22 and 23.

In certain aspects of this embodiment, the ficolin-based engineered MTMs comprise a ficolin microbe-binding domain comprising the fibrinogen-like domain of any one of SEQ ID NOs: 21, 22 and 23 or a sequence variant thereof having at least 85% sequence identity to any one of SEQ ID NOs: 21, 22 and 23 and an immunoglobulin domain comprising the amino acid sequence of SEQ ID NO:12 or a sequence variant thereof having at least 85% sequence identity to SEQ ID NO:12.

In a third embodiment, the invention is directed to compositions comprising toll-like receptor (TLR)-based MTMs. The TLR-based MTMs are either naturally-occurring TLR proteins or engineered MTM fusion proteins that comprise at least one TLR microbe-binding domain and at least one additional domain.

The naturally-occurring TLR protein may be any one of (i) TLR1, (ii) TLR2, (iii) TLR3, (iv) TLR4, (v) TLR5, (vi) TLR6, (vii) TLR7, (viii) TLR8, (ix) TLR9, (x) TLR10, and (xi) a sequence variant having at least 85% sequence identity to any one of (i)-(x).

The TLR microbe-binding domain of the TLR-based engineered MTMs may comprise the N-terminal ligand-binding domain of a TLR. The TLR may be any one of (i) TLR1, (ii) TLR2, (iii) TLR3, (iv) TLR4, (v) TLRS, (vi) TLR6, (vii) TLR7, (viii) TLR8, (ix) TLR9, (x) TLR10, and (xi) a sequence variant having at least 85% sequence identity to any one of (i)-(x).

The at least one additional domain of the TLR-based engineered MTMs may be one or more of (xii) a TLR transmembrane helix, (xiii) a TLR C-terminal cytoplasmic signaling domain, (xiv) a ficolin short N-terminal domain, (xv) a ficolin collagen-like domain, (xvi) a collectin cysteine-rich domain, (xvii) a collectin collagen-like domain, (xviii) a collectin coiled-coil neck domain, (xix) an oligomerization domain, (xx) a signal domain, (xxi) an anchor domain, (xxii) a collagen-like domain, (xxiii) a fibrinogen-like domain, (xxiv) an immunoglobulin domain, (xxv) an immunoglobulin-like domain, and (xxvi) a sequence variant having at least 85% sequence identity to any one of (xii)-(xxv).

In certain aspects of this embodiment, the at least one additional domain is an immunoglobulin domain. For example, the immunoglobulin domain may comprise the amino acid sequence of SEQ ID NO:12 or a sequence variant thereof having at least 85% sequence identity to SEQ ID NO:12.

In certain aspects of this embodiment, the TLR-based engineered MTMs comprise a TLR microbe-binding domain comprising the N-terminal ligand-binding domain of any one of SEQ ID NOs:24-33 or a sequence variant thereof having at least 85% sequence identity to any one of SEQ ID NOs:24-33.

In certain aspects of this embodiment, the TLR-based engineered MTMs comprise a TLR microbe-binding domain comprising the N-terminal ligand-binding domain of any one of SEQ ID NOs:24-33 or a sequence variant thereof having at least 85% sequence identity to any one of SEQ NOs:24-33 and an immunoglobulin domain comprising the amino acid sequence of SEQ ID NO:12 or a sequence variant thereof having at least 85% sequence identity to SEQ ID NO:12.

In a fourth embodiment, the invention is directed to a composition comprising one or more of the MTMs of the invention. In certain aspects of this embodiment, the composition comprises at least one naturally-occurring MTM. In certain other aspects of this embodiment, the composition comprises one collectin-based engineered MTM, wherein the collectin-based engineered MTM is an FcMBL of SEQ ID NO:6, 7, 8 or 9, or a sequence variant thereof having at least 85% sequence identity to SEQ ID NO:6, 7, 8 or 9. In certain other aspects of this embodiment, the composition comprises at least two collectin-based engineered MTMs, wherein one of the collectin-based engineered MTMs is an FcMBL of SEQ ID NO:6, 7, 8 or 9, or a sequence variant thereof having at least 85% sequence identity to SEQ ID NO:6, 7, 8 or 9. In certain other aspects of this embodiment, the composition comprises one naturally-occurring collectin MTM (e.g. MBL) and one collectin-based engineered MTM, wherein the collectin-based engineered MTM is an FcMBL of SEQ ID NO:6, 7, 8 or 9, or a sequence variant thereof having at least 85% sequence identity to SEQ ID NO:6, 7, 8 or 9. In certain other aspects of this embodiment, the composition comprises one naturally-occurring collectin MTM (e.g. MBL) and at least two collectin-based engineered MTMs, wherein one of the collectin-based engineered MTMs is an FcMBL of SEQ ID NO:6, 7, 8 or 9, or a sequence variant thereof having at least 85% sequence identity to SEQ ID NO:6, 7, 8 or 9.

In certain aspects of this embodiment, the composition further comprises a carrier or diluent, such as an aqueous solution comprising sodium acetate having a pH of about 3.2

In a fifth embodiment, the invention is directed to a composition according to any of the preceding embodiments, further comprising a therapeutic agent, for example, one or more antimicrobial agents.

### Devices

The MTMs and compositions of the invention may be used in devices (and related methods) to detect, capture and/or identify microbes and microbial components in a sample or in the environment.

The devices of the present invention generally comprise a substrate onto which a composition comprising MTMs of the invention is applied. Thus, and in a sixth embodiment, the invention is directed to a device comprising MTMs as defined herein.

In some aspects, a composition comprising MTMs can be coated directly onto the device comprising a substrate. In some aspects, a composition comprising MTMs can be coated onto a substrate and then affixed to the device. In some aspects, the device comprises a garment coated with a composition comprising MTMs of the present invention, for example a face covering such as a mask, or any type of outerwear such as a shirt, pants, a hat, and the like.

In some other aspects, the device comprises a swab coated with a composition comprising MTMs of the invention, for example, a nasal or oral swab that can be used to apply the composition comprising MTMs to the nose or mouth, respectively. A coated swab can also be used as a collection device for collecting and analyzing a sample, e.g., detecting and/or identifying one or more pathogens. In some aspects, a sample can be collected directly from a subject, e.g. using a coated swab to collect a fluid from the nose, mouth or eye(s). The advantage of a coated collection device is that the composition, particularly the at least one MTM portion thereof, concentrates the sample for subsequent analysis. In some aspects, a sample can be collected directly from a garment worn by the subject. In some aspects, the device comprises a bandage, such as a cloth or paper bandage, a liquid bandage, a hydrogel bandage, a foam bandage, or an adhesive bandage.

A sample can include but is not limited to, a patient sample, an animal or animal model sample, an agricultural sample, a food and beverage sample, an environmental sample, a pharmaceutical sample, a biological sample, and a non-biological sample. A biological sample can include but is not limited to, cells, tissue, peripheral blood, and a bodily fluid. Exemplary biological samples include, but are not limited to, a biopsy, a tumor sample, biofluid sample; blood; serum; plasma; urine; feces; sperm; mucus; tissue biopsy; organ biopsy; synovial fluid; bile fluid; cerebrospinal fluid; mucosal secretion; effusion; sweat; saliva; and/or tissue sample etc. The sample can be collected from any source, including, e.g., human, animal, plant, environment, and organic or inorganic materials, suspected of being infected or contaminated by a microbe(s). Environmental samples include, but are not limited to, air samples, liquid samples, and dry samples. Suitable air samples include, but are not limited to, an aerosol, an atmospheric sample, and a ventilator discharge. Suitable dry samples include, but are not limited to, soil. Pharmaceutical samples include, but are not limited to, drug material samples and therapeutic fluid samples, for example, for quality control or detection of endotoxins. Suitable therapeutic fluids include, but are not limited to, a dialysis fluid.

In a seventh embodiment, the invention is directed to methods of using the devices of the invention. Such methods include applying a device or substrate comprising a composition comprising MTMs to a subject (e.g., a human or an animal subject). In some aspects, applying a device or substrate to a subject can include preventing one or more pathogens from entering a subject, e.g., into the eye(s), nose, mouth, and/or respiratory system, including the airway, lungs and blood vessels, and blood, for example, where the device is a garment worn by the subject, e.g. a mask over the nose and mouth. In some aspects, applying a device or substrate to a subject can include preventing one or more pathogens from entering a subject, e.g., into a wound, for example, where the device is a bandage worn by the subject. In some aspects, applying a device or substrate to a subject can include collecting a sample from a subject, for example, where the device is a collection device such as a cotton swab. In any of these aspects, the method can further comprise any one of: capturing one or more microbe or microbe components; collecting and preparing a sample for analysis; and analyzing the sample, e.g., detecting and identifying one or more pathogens.

The invention is also directed to methods of detecting a microbe in a sample, comprising contacting a sample suspected of containing a microbe with a device or substrate of the invention comprising MTMs under conditions permitting binding of a microbe or a component of a microbe by MTMs coated on, or otherwise displayed by, the device or substrate, thereby detecting a microbe in a sample.

The invention is also directed to methods of detecting a microbial infection in a subject, comprising contacting a biological sample of a subject suspected of having a microbial infection with a device or substrate of the invention comprising MTMs under conditions permitting binding of a microbe or a component of a microbe by MTMs coated on, or otherwise displayed by, the device or substrate, thereby detecting a microbial infection in the subject.

The invention is also directed to methods of diagnosing a microbial infection in a subject, comprising contacting a biological sample of a subject suspected of having a microbial infection with a device or substrate of the invention comprising MTMs under conditions permitting binding of a microbe or a component of a microbe by MTMs coated on, or otherwise displayed by, the device or substrate, thereby diagnosing a microbial infection in the subject.

The invention is also directed to methods of treating a microbial infection in a subject, comprising contacting a bodily fluid of a subject having a microbial infection with a device or substrate of the invention comprising MTMs under conditions permitting binding of a microbe or a component of a microbe by MTMs coated on, or otherwise displayed by, the device or substrate, thereby treating a microbial infection in the subject.

The invention is also directed to methods of filtering a microbe or microbial component from a fluid, comprising contacting a fluid containing a microbe or microbial component with a device or substrate of the invention comprising MTMs under conditions permitting binding of a microbe or a component of a microbe by MTMs coated on, or otherwise displayed by, the device or substrate, thereby filtering a microbe or microbial component from a fluid.

The invention is also directed to methods of inhibiting or preventing entry of a microbe or microbial component into a subject, comprising applying a device or substrate comprising MTMs of the invention to a subject. In certain aspects, the device is a mask covering the mouth and nose of the subject. In certain other aspects, the device is a bandage covering a wound of the subject.

In each of these methods, the methods may further comprise identifying the microbe bound by the MTMs.

In each of these methods, the sample may be a biological sample. For example, the biological sample may be blood.

In each of these methods, the device may comprise two or more MTMs having different binding specificities.

### BRIEF DESCRIPTION OF THE DRAWINGS

FIG. 1. Diagram showing the structure of exemplary collectins and ficolins.

FIG. 2. Diagram showing a device for diagnosing and/or treating infectious disease comprising a collection swab.

FIG. 3. Diagram showing a device for diagnosing and/or treating infectious disease comprising a toothbrush.

### DETAILED DESCRIPTION

### I. Definitions

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. The Present Invention

As summarized above, the present invention is generally directed to compositions, devices and methods for capturing, detecting and/or identifying one or more microbes or microbe components and/or treating or preventing infectious disease in a subject (e.g., a human or an animal subject) using microbe-targeting molecules (MTMs) and engineered MTMs as defined herein.

The devices of the invention can be used, for example, in the detection and/or identification of microbes in a biological sample, and alternatively, or in addition in the prevention of microbes from entering a subject.

The important characteristic of the MTMs used in the compositions, devices and methods of the invention is that these constructs contact and bind microbes and microbial components in a sample based on the identity of the MAMP produced by the microbe, rather than the identity of microbe itself. While some MAMPs are produced by only a single species of microbe, other MAMPs are shared across species. Thus, while some MTMs of the invention bind to only MAMPs of a particular species of microbe, other MTMs of the invention can bind to MAMPs produced by all members of a particular class, order, family, genus or sub-genus of microbe.

As used herein, “MTM” and “engineered MTM” refers to any of the molecules described herein (or described in patents or patent application incorporated by reference) that can bind to a microbe or microbe component. Unless the context indicates otherwise, the term “MTM” is used to describe all MTMs of the invention, both naturally-occurring and engineered forms of these constructs. The terms “microbe-targeting molecule” and “microbe-binding molecule” are used interchangeably herein.

### MAMPs

Before discussing the MTMs of the invention, it will be helpful to understand the molecules to which the MTMs bind. As indicated above, each of the MTMs of the invention binds to at least one microbe-associated molecular pattern (MAMP). Some MTMs bind at least two, at least three, at least four, at least five, or more than five MAMPs.

As used herein and throughout the specification, the term “microbe-associated molecular patterns” or “MAMPs” refers to molecules, components or motifs associated with or secreted or released by microbes or groups of microbes (whole and/or lysed and/or disrupted) that are generally recognized by corresponding pattern recognition receptors (PRRs) of the MTM microbe-binding domains defined herein. In some aspects, the MAMPs encompass molecules associated with cellular components released during cell damage or lysis. Examples of MAMPs include, but are not limited to, microbial carbohydrates (e.g., lipopolysaccharide or LPS, mannose), endotoxins, microbial nucleic acids (e.g., bacterial, fungal or viral DNA or RNA; e.g., nucleic acids comprising a CpG site), microbial peptides (e.g., flagellin), peptidoglycans, lipoteichoic acids, N-formylmethionine, lipoproteins, lipids, phospholipids or their precursors (e.g., phosphochloline), and fungal glucans.

In some aspects, microbe components comprise cell wall or membrane components known as pathogen-associated molecular patterns (PAMPs) including lipopolysaccharide (LPS) endotoxin, lipoteichoic acid, and attached or released outer membrane vesicles. In some aspects, a microbe comprises a host cell membrane and a pathogen component or a PAMP.

In some aspects, microbe components comprise damage-associated molecular patterns (DAMPs), also known as danger-associated molecular patterns, danger signals, and alarmin. These biomolecules can initiate and sustain a non-infectious inflammatory response in a subject, in contrast to PAMPs which initiate and sustain an infectious pathogen-induced inflammatory response. Upon release from damaged or dying cells, DAMPs activate the innate immune system through binding to pattern recognition receptors (PRRs). DAMPs are recognized by immune receptors, such as toll-like receptors (TLRs) and NOD-like receptor family, pyrin domain containing 3 (NLRP3), expressed by sentinel cells of the immune system. DAMPs include portions of nuclear and cytosolic proteins, ECM (extracellular matrix), mitochondria, granules, ER (endoplasmic reticulum), and plasma membrane.

In some aspects, MAMPs include carbohydrate recognition domain (CRD)-binding motifs. As used herein, the term “carbohydrate recognition domain (CRD)-binding motifs” refers to molecules or motifs that are bound by a molecule or composition comprising a CRD (i.e. CRDs recognize and bind to CRD-binding motifs). As used herein, the term “carbohydrate recognition domain” or “CRD” refers to one or more regions, at least a portion of which, can bind to carbohydrates on a surface of microbes or pathogens. In some aspects, the CRD can be derived from a lectin, as described herein. In some aspects, the CRD can be derived from a mannan-binding lectin (MBL). Accordingly, in some aspects, MAMPs are molecules, components or motifs associated with microbes or groups of microbes that are recognized by lectin-based MTMs (collectin-based MTMs) described herein that have a CRD domain. In one embodiment, MAMPs are molecules, components, or motifs associated with microbes or groups of microbes that are recognized by mannan-binding lectin (MBL).

In some aspects, MAMPs are molecules, components or motifs associated with microbes or groups of microbes that are recognized by a C-reactive protein (CRP)-based MTMs (collectin-based MTMs).

For clarity, MAMPs as used herein includes microbe components such as MAMPs, PAMPs and DAMPs as defined above.

When necessary, and unless otherwise detectable without pre-treatment, MAMPs can be exposed, released or generated from microbes in a sample by various sample pretreatment methods. In some aspects, the MAMPs can be exposed, released or generated by lysing or killing at least a portion of the microbes in the sample. Without limitations, any means known or available to the practitioner for lysing or killing microbe cells can be used. Exemplary methods for lysing or killing the cells include, but are not limited to, physical, mechanical, chemical, radiation, biological, and the like. Accordingly, pre-treatment for lysing and/or killing the microbe cells can include application of one or more of ultrasound waves, vortexing, centrifugation, vibration, magnetic field, radiation (e.g., light, UV, Vis, IR, X-ray, and the like), change in temperature, flash-freezing, change in ionic strength, change in pH, incubation with chemicals (e.g. antimicrobial agents), enzymatic degradation, and the like.

### Microbes

As used herein, the term “microbe”, and the plural “microbes”, generally refers to microorganism(s), including bacteria, virus, fungi, parasites, protozoan, archaea, protists, e.g., algae, and a combination thereof. The term “microbe” encompasses both live and dead microbes. The term “microbe” also includes pathogenic microbes or pathogens, e.g., bacteria causing diseases such as sepsis, plague, tuberculosis and anthrax; protozoa causing diseases such as malaria, sleeping sickness and toxoplasmosis; and fungi causing diseases such as ringworm, candidiasis or histoplasmosis.

In some aspects, the microbe is a human pathogen, in other words a microbe that causes at least one disease in a human.

In some aspects, the microbe is a Gram-positive bacterial species, a Gram-negative bacterial species, a mycobacterium, a fungus, a parasite, protozoa, or a virus. In some aspects, the Gram-positive bacterial species comprises bacteria from the class Bacilli. In some aspects, the Gram-negative bacterial species comprises bacteria from the class Gammaproteobacteria. In some aspects, the mycobacterium comprises bacteria from the class Actinobacteria. In some aspects, the fungus comprises fungus from the class Saccharomycetes.

In some aspects, the microbe is Staphylococcus aureus, Streptococcus pyogenes, Klebsiella pneumoniae, Pseudomonas aeruginosa, Mycobacterium tuberculosis, Candida albicans, or Escherichia coli. In some aspects, the microbe is S. aureus strain 3518, S. pyogenes strain 011014, K. pneumoniae strain 631, E. coli strain 41949, P. aeruginosa strain 41504, C. albicans strain 1311, or M. tuberculosis strain H37Rv.

In some aspects, the microbe is Bartonella henselae, Borrelia burgdorferi, Campylobacter jejuni, Campylobacterfetus, Chlamydia trachomatis, Chlamydia pneumoniae, Chylamydia psittaci, Simkania negevensis, Escherichia coli (e.g., 0157:H7 and K88), Ehrlichia chafeensis, Clostridium botulinum, Clostridium perfringens, Clostridium tetani, Enterococcus faecalis, Haemophilius influenzae, Haemophilius ducreyi, Coccidioides immitis, Bordetella pertussis, Coxiella burnetii, Ureaplasma urealyticum, Mycoplasma genitalium, Trichomatis vaginalis, Helicobacter pylori, Helicobacter hepaticus, Legionella pneumophila, Mycobacterium tuberculosis, Mycobacterium bovis, Mycobacterium africanum, Mycobacterium leprae, Mycobacterium asiaticum, Mycobacterium avium, Mycobacterium celatum, Mycobacterium celonae, Mycobacterium fortuitum, Mycobacterium genavense, Mycobacterium haemophilum, Mycobacterium intracellulare, Mycobacterium kansasii, Mycobacterium malmoense, Mycobacterium marinum, Mycobacterium scrofulaceum, Mycobacterium simiae, Mycobacterium szulgai, Mycobacterium ulcerans, Mycobacterium xenopi, Corynebacterium diptheriae, Rhodococcus equi, Rickettsia aeschlimannii, Rickettsia africae, Rickettsia conorii, Arcanobacterium haemolyticum, Bacillus anthracia, Bacillus cereus, Lysteria monocytogenes, Yersinia pestis, Yersinia enterocolitica, Shigella dysenteriae, Neisseria meningitides, Neisseria gonorrhoeae, Streptococcus bovis, Streptococcus hemolyticus, Streptococcus mutans, Streptococcus pyogenes, Streptococcus pneumoniae, Staphylococcus aureus, Staphylococcus epidermidis, Staphylococcus pneumoniae, Staphylococcus saprophyticus, Vibrio cholerae, Vibrio parahaemolyticus, Salmonella typhi, Salmonella paratyphi, Salmonella enteritidis, Treponema pallidum, Human rhinovirus, Human coronavirus such as SARS-CoV-2, Dengue virus, Filoviruses (e.g., Marburg and Ebola viruses), Hantavirus, Rift Valley virus, Hepatitis B, C, and E, Human Immunodeficiency Virus (e.g., HIV-1, HIV-2), HHV-8, Human papillomavirus, Herpes virus (e.g., HV-I and HV-II), Human T-cell lymphotrophic viruses (e.g., HTLV-I and HTLV-II), Bovine leukemia virus, Influenza virus, Guanarito virus, Lassa virus, Measles virus, Rubella virus, Mumps virus, Chickenpox (Varicella virus), Monkey pox, Epstein Bahr virus, Norwalk (and Norwalk-like) viruses, Rotavirus, Parvovirus B19, Hantaan virus, Sin Nombre virus, Venezuelan equine encephalitis, Sabia virus, West Nile virus, Yellow Fever virus, causative agents of transmissible spongiform encephalopathies, Creutzfeldt-Jakob disease agent, variant Creutzfeldt-Jakob disease agent, Candida, Cryptcooccus, Cryptosporidium, Giardia lamblia, Microsporidia, Plasmodium vivax, Pneumocystis carinii, Toxoplasma gondii, Trichophyton mentagrophytes, Enterocytozoon bieneusi, Cyclospora cayetanensis, Encephalitozoon hellem, or Encephalitozoon cuniculi, among other viruses, bacteria, archaea, protozoa, and fungi. In yet other aspects, the microbe is a bioterror agent (e.g., B. anthracis, and smallpox).

As used herein, “microbe component” and “microbial component” refer to any part of a microbe such as cell wall components, cell membrane components, cell envelope components, cytosolic components, intracellular components, nucleic acid (DNA or RNA), or organelles in the case of eukaryotic microbes. The terms “microbe component” and “microbial component” have the same meaning and they are used interchangeably herein. In some aspects, the microbial component comprises a component from a Gram-positive bacterial species, a Gram-negative bacterial species, a mycobacterium, a fungus, a parasite, a virus, or any microbe described herein or known in the art.

### Sample

The MTMs defined herein can be used to detect, isolate and/or identify a microbe or a microbial component in a sample or in the environment. A sample can include but is not limited to, a patient sample, an animal or animal model sample, an agricultural sample, a food and beverage sample, an environmental sample, a pharmaceutical sample, a biological sample, and a non-biological sample. A biological sample can include but is not limited to, cells, tissue, peripheral blood, and a bodily fluid. Exemplary biological samples include, but are not limited to, a biopsy, a tumor sample, biofluid sample; blood; serum; plasma; urine; sperm; mucus; tissue biopsy; organ biopsy; synovial fluid; bile fluid; cerebrospinal fluid; mucosal secretion; effusion; sweat; saliva; and/or tissue sample etc. The biological sample can be collected from any source, including, e.g., human or animal suspected of being infected or contaminated by a microbe(s). Biological fluids can include a bodily fluid and may be collected in any clinically acceptable manner. Biological fluids can include, but are not limited to, mucous, phlegm, saliva, sputum, blood, plasma, serum, serum derivatives, bile, sweat, amniotic fluid, menstrual fluid, mammary fluid, peritoneal fluid, interstitial fluid, urine, semen, synovial fluid, interocular fluid, a joint fluid, an articular fluid, and cerebrospinal fluid (CSF). A fluid may also be a fine needle aspirate or biopsied tissue. Blood fluids can be obtained by standard phlebotomy procedures and may be separated into components such as plasma for analysis. Centrifugation can be used to separate out fluid components to obtain plasma, buffy coat, erythrocytes, cells, pathogens and other components.

In some aspects, the sample, such as a fluid, may be purified before introduction to a device or a system of the invention. For example, filtration or centrifugation to remove particulates and chemical interference may be used. Various filtration media for removal of particles includes filter paper, such as cellulose and membrane filters, such as regenerated cellulose, cellulose acetate, nylon, PTFE, polypropylene, polyester, polyethersulfone, polyarylethersulfone, polycarbonate, and polyvinylpyrolidone.

Environmental samples include, but are not limited to, air samples, liquid and fluid samples, and dry samples. Suitable air samples include, but are not limited to, an aerosol, an atmospheric sample, and a ventilator discharge. Suitable dry samples include, but are not limited to, soil. Environmental fluids include, for example, saturated soil water, groundwater, surface water, unsaturated soil water; and fluids from industrialized processes such as waste water. Agricultural fluids can include, for example, crop fluids, such as grain and forage products, such as soybeans, wheat, and corn.

Pharmaceutical samples include, but are not limited to, drug material samples and therapeutic fluid samples, for example, for quality control or detection of endotoxins. Suitable therapeutic fluids include, but are not limited to, a dialysis fluid.

Further, reference to “in the environment” simply means the environs in which the subject is located, for example, the air surrounding the subject. Thus, a subject wearing a device of the invention, such as a garment mask to which a composition comprising MTMs has been applied, may be protected from inhaling microbes as the subject goes about daily life. As the subject breathes and air passes through the mask, MTMs on the mask can bind and isolate the microbes from the air before the air enters the subject's mouth or nose.

### Subject

The present invention, including the devices, systems and methods, may be used in conjunction with a subject. For example, the therapeutic devices may be used in the treatment of a subject, and the systems of the invention may be used to filter pathogens from the blood of a subject. As used herein, a “subject” is a human, a non-human primate, bird, horse, cow, goat, sheep, a companion animal, such as a dog, cat or rodent, or other mammal. The subject may be a patient undergoing treatment for a medical condition.

### MTMs

As summarized above, the invention is directed to compositions and devices, and methods of using the devices, where the devices comprise MTMs that bind to one or more MAMPs. The devices of the invention can be used, for example, in the detection, isolation and/or identification of microbes and microbe components in a sample or in the environment.

MTMs distinguish and bind microbes and microbial components from a sample or in the environment based on the identity of the MAMP produced by the microbe, rather than the identity of microbe itself. While some MAMPs are produced by only a single species of microbe, other MAMPs are shared across species. Thus, while some MTMs of the invention bind to only MAMPs of a particular species of microbe, other MTMs of the invention can bind to MAMPs produced by all members of a particular class, order, family, genus or sub-genus of microbe.

As will be apparent, while the “MTMs” of the invention include naturally-occurring molecules and proteins, the “engineered MTMs” of the invention are those have been manipulated in some manner by the hand of man. As used herein and throughout the specification, the term “engineered MTM” includes any non-naturally-occurring MTM. Engineered MTMs of the invention retain the binding specificity to a MAMP of the wild-type (i.e. naturally-occurring) molecule on which the engineered MTM is based.

The MTMs of the invention are defined based on their binding activity, therefore both naturally-occurring and engineered MTMs will comprise at least one microbe-binding domain, i.e. a domain that recognizes and binds to one or more MAMPs (including, at least two, at least three, at least four, at least five, or more) as described herein. A microbe-binding domain can be a naturally-occurring or a synthetic molecule. In some aspects, a microbe-binding domain can be a recombinant molecule.

Acceptable microbe-binding domains for use in the MTMs of the invention are limited only in their ability to recognize and bind at least one MAMP. In some aspects, the microbe-binding domain may comprise some or all of a peptide; polypeptide; protein; peptidomimetic; antibody; antibody fragment; antigen-binding fragment of an antibody; carbohydrate-binding protein; lectin; glycoprotein; glycoprotein-binding molecule; amino acid; carbohydrate (including mono-; di-; tri- and poly-saccharides); lipid; steroid; hormone; lipid-binding molecule; cofactor; nucleoside; nucleotide; nucleic acid; DNA; RNA; analogues and derivatives of nucleic acids; peptidoglycan; lipopolysaccharide; small molecule; endotoxin; bacterial lipopolysaccharide; and any combination thereof.

In particular aspects, the microbe-binding domain can be a microbe-binding domain of a lectin. An exemplary lectin is mannan binding lectin (MBL) or other mannan binding molecules. Non-limiting examples of acceptable microbe-binding domains also include microbe-binding domains from toll-like receptors, nucleotide oligomerization domain-containing (NOD) proteins, complement receptors, collectins, ficolins, pentraxins such as serum amyloid and C-reactive protein, lipid transferases, peptidoglycan recognition proteins (PGRs), and any combinations thereof. In some aspects, microbe-binding domains can be microbe-binding molecules described in the International Patent Application No. WO 2013/012924, the contents of which are incorporated by reference in their entirety.

The MTMs of the invention will typically have one or more domains in addition to a microbe-binding domain. Such domains include, but are not limited to, an oligomerization domain, a signal domain, an anchor domain, a collagen-like domain, a fibrinogen-like domain, an immunoglobulin domain, and an immunoglobulin-like domain.

Engineered MTMs of the invention include, but are not limited to, MTMs identical to a naturally-occurring MTM but having at least one amino acid change in comparison to the wild-type molecule on which they are based. Such “sequence-variant engineered MTMs” have at least 51, 52, 53, 54, 55, 56, 57, 58, 59, 60, 60, 61, 62, 63, 64, 65, 66, 67, 68, 69, 70, 71, 72, 73, 74, 76, 77, 78, 79, 80, 81, 82, 83, 84, 85, 86, 87, 88, 89, 90, 91, 92, 93, 94, 95, 96, 97, 98 or 99 sequence identity, though in all cases less than 100% sequence identity, to the wild-type molecule on which they are based. The changes may be any combination of additions, insertions, deletions and substitutions, where the altered amino acids may be naturally-occurring or non-naturally-occurring amino acids, and conservative or non-conservative changes.

Engineered MTMs of the invention also include, but are not limited to, MTMs that comprise domains from two or more different MTMs, i.e. fusion proteins. Such “domain-variant engineered MTMs” have domains from 2, 3, 4, 5 or more different proteins. For example, MTMs can be a fusion protein comprising a microbe-binding domain and an oligomerization domain, or a fusion protein comprising a microbe-binding domain and a signal domain, or a fusion protein comprising a microbe-binding domain, an oligomerization domain, and signal domain, to name a few examples. In each case, the domains within a domain-variant engineered MTM are from at least two different proteins. Other examples of such MTMs include fusion proteins comprising at least the microbe-binding domain of a lectin and at least a part of a second protein or peptide, e.g., but not limited, to an Fc portion of an immunoglobulin.

Engineered MTMs of the invention further include, but are not limited to, MTMs that comprise domains from two or more different MTMs, wherein at least one of the domains is a sequence variant of the wild-type domain upon which it is based, i.e. having at least one amino acid change in comparison to the wild-type molecule on which it is based. These “sequence- and domain-variant engineered MTMs” have domains from 2, 3, 4, 5 or more different proteins, and at least one of the domains has at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 91, 92, 93, 94, 95, 96, 97, 98 or 99 sequence identity, though in all cases less than 100% sequence identity, to the wild-type domain on which it is based. The changes may be any combination of additions, insertions, deletions and substitutions, where the altered amino acids may be naturally-occurring or non-naturally-occurring amino acids, and conservative or non-conservative changes.

As non-limiting examples of the MTMs of the invention, three broad categories of suitable MTMs are defined in the following paragraphs, namely: (i) collectin-based MTMs, (ii) ficolin-based MTMs, and (iii) toll-like receptor-based MTMs. It should be understood that these three categories are not the only categories of MTMs encompassed by the invention.

### Collectin-Based MTMs

The MTMs of the invention include collectin-based MTMs. These MTMs are either naturally-occurring collectin proteins or engineered MTM fusion proteins that comprise at least one collectin microbe-binding domain, such as the lectin carbohydrate-recognition domain (CRD), and at least one additional domain

Collectins (collagen-containing C-type lectins) are a family of collagenous calcium-dependent lectins that function in defense, thus playing an important role in the innate immune system. They are soluble molecules comprising pattern recognition receptors (PRRs) within the microbe-binding domain that recognize and bind to particular oligosaccharide structures or lipids displayed on the surface of microbes, i.e. MAMPs of oligosaccharide origin. Upon binding of collectins to a microbe, clearance of the microbe is achieved via aggregation, complement activation, opsonization, and activation of phagocytosis.

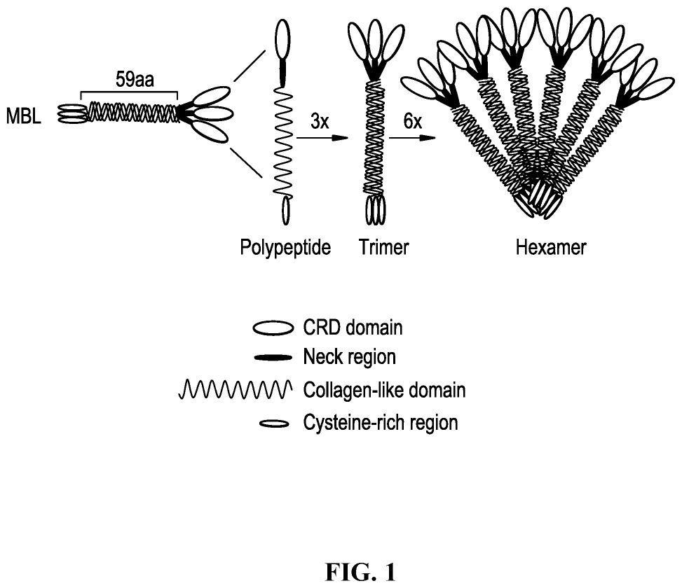

Members of the family have a common structure, characterized by four parts or domains arranged in the following N- to C-terminal arrangement: (i) a cysteine-rich domain, (ii) a collagen-like domain, (iii) a coiled-coil neck domain, and (iv) a microbe-binding domain which includes a C-type lectin domain, also termed the carbohydrate recognition domain (CRD). The functional form of the molecule is a trimer made up of three identical chains. MAMP recognition is mediated by the CRD in presence of calcium. See FIG. 1.

There are currently nine recognized members of the family: (i) mannose-binding lectin (MBL; mannose-binding lectin; e.g. SEQ ID NO:1), (ii) surfactant protein A (SP-A; SEQ ID NO:13), (iii) surfactant protein D (SP-D; SEQ ID NO:14), (iv) collectin liver 1 (CL-Ll; SEQ ID NO:15), (v) collectin placenta 1 (CL-P1; SEQ ID NO:16), (vi) conglutinin collectin of 43 kDa (CL-43; SEQ ID NO:17), (vii) collectin of 46 kDa (CL-46; SEQ ID NO:18), (viii) collectin kidney 1 (CL-K1; SEQ ID NO:19), and (ix) conglutinin (SEQ ID NO:20). Each of these proteins is an MTM of the invention.

MBL (Homo sapiens; GenBank: AAH69338.1; SEQ ID NO: 1):
MSLFPSLPLL LLSMVAASYS ETVTCEDAQK TCPAVIACSS PGINGFPGKD
GRDGTKGEKG EPGQGLRGLQ GPPGKLGPPG NPGPSGSPGP KGQKGDPGKS
PDGDSSLAAS ERKALQTEMA RIKKWLTFSL GKQVGNKFFL TNGEIMTFEK
VKALCVKFQA SVATPRNAAE NGAIQNLIKE EAFLGITDEK TEGQFVDLTG
NRLTYTNWNE GEPNNAGSDE DCVLLLKNGQ WNDVPCSTSH LAVCEFPI
Surfactant protein A (SP-A; Homo sapiens; GenBank: AAA36632.1;
SEQ ID NO: 13):
MTFKNNTTLS LFRLFNSFPL HKGYITTREL HQLALANLIF MFNCADLHFP
PQMRKCFPIP GTSCLFCPKT KTLFPVLHLA NQYLCFLCQH KHCFPQTRFP
DSQPSQATSR CVLSIKFTSS IYINSHNYTL ILTCIIVRVL CPPVGCKSYK
DRGHIHLAHP SVPSTCHGSY QLTSTQYIVW ISKYDSNFQH PKLVYSPTEP
AQVEHMQCFL CMCLQREERE ALLLLPRVTI LTRLSAESTD ERDGDSEPVN
AVCRTALAFV PHESNVMLGI HNLLIWLL
Surfactant protein D (SP-D; Homo sapiens; GenBank: AAB59450.1;
SEQ ID NO: 14):
MLLFLLSALV LLTQPLGYLE AEMKTYSHRT MPSACTLVMC SSVESGLPGR
DGRDGREGPR GEKGDPGLPG AAGQAGMPGQ AGPVGPKGDN GSVGEPGPKG
DTGPSGPPGP PGVPGPAGRE GALGKQGNIG PQGKPGPKGE AGPKGEVGAP
GMQGSAGARG LAGPKGERGV PGERGVPGNT GAAGSAGAMG PQGSPGARGP
PGLKGDKGIP GDKGAKGESG LPDVASLRQQ VEALQGQVQH LQAAFSQYKK
VELFPNGQSV GEKIFKTAGF VKPFTEAQLL CTQAGGQLAS PRSAAENAAL
QQLVVAKNEA AFLSMTDSKT EGKFTYPTGE SLVYSNWAPG EPNDDGGSED
CVEIFTNGKW NDRACGEKRL VVCEF
Collectin liver 1 (CL-L1; also known as collectin-10;
Homo sapiens; NCBI Reference Sequence: NP_006429.2;
SEQ ID NO: 15):
MNGFASLLRR NQFILLVLFL LQIQSLGLDI DSRPTAEVCA THTISPGPKG
DDGEKGDPGE EGKHGKVGRM GPKGIKGELG DMGDQGNIGK TGPIGKKGDK
GEKGLLGIPG EKGKAGTVCD CGRYRKFVGQ LDISIARLKT SMKFVKNVIA
GIRETEEKFY YIVQEEKNYR ESLTHCRIRG GMLAMPKDEA ANTLIADYVA
KSGFFRVFIG VNDLEREGQY MFTDNTPLQN YSNWNEGEPS DPYGHEDCVE
MLSSGRWNDT ECHLTMYFVC EFIKKKK
Collectin placenta 1 (CL-P1; Homo sapiens; GenBank: BAB72147.1;
SEQ ID NO: 16):
MKDDFAEEEE VQSFGYKRFG IQEGTQCTKC KNNWALKFSI ILLYILCALL
TITVAILGYK VVEKMDNVTG GMETSRQTYD DKLTAVESDL KKLGDQTGKK
AISTNSELST FRSDILDLRQ QLREITEKTS KNKDTLEKLQ ASGDALVDRQ
SQLKETLENN SFLITTVNKT LQAYNGYVTN LQQDTSVLQG NLQNQMYSHN
VVIMNLNNLN LTQVQQRNLI TNLQRSVDDT SQAIQRIKND FQNLQQVFLQ
AKKDTDWLKE KVQSLQTLAA NNSALAKANN DTLEDMNSQL NSFTGQMENI
TTISQANEQN LKDLQDLHKD AENRTAIKFN QLEERFQLFE TDIVNIISNI
SYTAHHLRTL TSNLNEVRTT CTDTLTKHTD DLTSLNNTLA NIRLDSVSLR
MQQDLMRSRL DTEVANLSVI MEEMKLVDSK HGQLIKNFTI LQGPPGPRGP
RGDRGSQGPP GPTGNKGQKG EKGEPGPPGP AGERGPIGPA GPPGERGGKG
SKGSQGPKGS RGSPGKPGPQ GPSGDPGPPG PPGKEGLPGP QGPPGFQGLQ
GTVGEPGVPG PRGLPGLPGV PGMPGPKGPP GPPGPSGAVV PLALQNEPTP
APEDNGCPPH WKNFTDKCYY FSVEKEIFED AKLFCEDKSS HLVFINTREE
QQWIKKQMVG RESHWIGLTD SERENEWKWL DGTSPDYKNW KAGQPDNWGH
GHGPGEDCAG LIYAGQWNDF QCEDVNNFIC EKDRETVLSS AL
Conglutinin collectin (CL-43; Bos taurus; GenBank: AAI16148;
SEQ ID NO: 17):
MLPLPLSILL LLTQSQSFLG EEMDVYSEKT LTDPCTVVVC APPADSLRGH
DGRDGKEGPQ GEKGDPGPPG MPGPAGREGP SGRQGSMGPP GTPGPKGEPG
PEGGVGAPGM PGSPGPTGLK GERGTPGPGG AIGPQGPSGA MGPPGLKGDR
GDPGEKGAKG ETSVLEVDTL RQRMRNLEGE VQRLQNIVTQ YRKAVLFPDG
QAVGEKIFKT AGAVKSYSDA EQLCREAKGQ LASPRSSAEN EAVTQLVRAK
NKHAYLSMND ISKEGKFTYP TGGSLDYSNW APGEPNNRAK DEGPENCLEI
YSDGNWNDIE CREERLVICE F
Collectin of 46 kDa (CL-46; Bos taurus; GenBank: AAM34742;
SEQ ID NO: 18):
MLLLPLSVLL LLTQPWRSLG AEMKIYSQKT LANGCTLVVC RPPEGGLPGR
DGQDGREGPQ GEKGDPGSPG PAGRAGRPGP AGPIGPKGDN GSAGEPGPKG
DTGPPGPPGM PGPAGREGPS GKQGSMGPPG TPGPKGDTGP KGGMGAPGMQ
GSPGPAGLKG ERGAPGELGA PGSAGVAGPA GAIGPQGPSG ARGPPGLKGD
RGDPGERGAK GESGLADVNA LKQRVTILEG QLQRLQNAFS RYKKAVLFPD
GQAVGKKIFK TAGAVKSYSD AQQLCREAKG QLASPRSAAE NEAVAQLVRA
KNNDAFLSMN DISTEGKFTY PTGESLVYSN WASGEPNNNN AGQPENCVQI
YREGKWNDVP CSEPLLVICE F
Collectin kidney 1 (CL-K1; also known as collectin-11;
Homo sapiens; GenBank: BAF43301.1; SEQ ID NO: 19):
MRGNLALVGV LISLAFLSLL PSGHPQPAGD DACSVQILVP GLKGDAGEKG
DKGAPGRPGR VGPTGEKGDM GDKGQKGSVG RHGKIGPIGS KGEKGDSGDI
GPPGPNGEPG LPCECSQLRK AIGEMDNQVS QLTSELKFIK NAVAGVRETE
SKIYLLVKEE KRYADAQLSC QGRGGTLSMP KDEAANGLMA AYLAQAGLAR
VFIGINDLEK EGAFVYSDHS PMRTFNKWRS GEPNNAYDEE DCVEMVASGG
WNDVACHTTM YFMCEFDKEN M
Conglutinin (Bos taurus; GenBank: BAA04983; SEQ ID NO: 20):
MLLLPLSVLL LLTQPWRSLG AEMTTFSQKI LANACTLVMC SPLESGLPGH
DGQDGRECPH GEKGDPGSPG PAGRAGRPGW VGPIGPKGDN GFVGEPGPKG
DTGPRGPPGM PGPAGREGPS GKQGSMGPPG TPGPKGETGP KGGVGAPGIQ
GFPGPSGLKG EKGAPGETGA PGRAGVTGPS GAIGPQGPSG ARGPPGLKGD
RGDPGETGAK GESGLAEVNA LKQRVTILDG HLRRFQNAFS QYKKAVLFPD
GQAVGEKIFK TAGAVKSYSD AEQLCREAKG QLASPRSSAE NEAVTQMVRA
QEKNAYLSMN DISTEGRFTY PTGEILVYSN WADGEPNNSD EGQPENCVEI
FPDGKWNDVP CSKQLLVICE F

The MTMs of the invention also include other collectin-based molecules that bind to one or more MAMPs, e.g. those MTMs comprising at least a portion (e.g. domain) of a lectin-based molecule in the case of an engineered MTM. As used herein, the term “collectin-based molecule” refers to a molecule comprising a microbe-binding domain derived from a collectin, such as a lectin. The term “lectin” as used herein refers to any molecule including proteins, natural or genetically modified (e.g., recombinant), that interacts specifically with saccharides (e.g., carbohydrates). The term “lectin” as used herein can also refer to lectins derived from any species, including, but not limited to, plants, animals (e.g. mammals, such as human), insects and microorganisms, having a desired carbohydrate binding specificity. Examples of plant lectins include, but are not limited to, the Leguminosae lectin family, such as ConA, soybean agglutinin, peanut lectin, lentil lectin, and Galanthus nivalis agglutinin (GNA) from the Galanthus (snowdrop) plant. Other examples of plant lectins are the Gramineae and Solanaceae families of lectins. Examples of animal lectins include, but are not limited to, any known lectin of the major groups S-type lectins, C-type lectins, P-type lectins, and I-type lectins, and galectins. In some aspects, the carbohydrate recognition domain can be derived from a C-type lectin, or a fragment thereof. C-type lectin can include any carbohydrate-binding protein that requires calcium for binding (e.g., MBL). In some aspects, the C-type lectin can include, but are not limited to, collectin, DC-SIGN, and fragments thereof. Without wishing to be bound by theory, DC-SIGN can generally bind various microbes by recognizing high-mannose-containing glycoproteins on their envelopes and/or function as a receptor for several viruses such as HIV and Hepatitis C.

Collectin-based engineered MTMs of the invention are MTMs that comprise at least a microbe-binding domain of a collectin. These MTMs may also include one or more of the other domains of a collectin, e.g. a cysteine-rich domain, a collagen-like domain, and/or a coiled-coil neck domain, as well as one or more domains not typically found in a collectin, such as an oligomerization domain, a signal domain, an anchor domain, a collagen-like domain, a fibrinogen-like domain, an immunoglobulin domain, and/or an immunoglobulin-like domain. When a collectin-based engineered MTM has each of the domains of a wild-type collectin, the MTM will be a sequence-variant engineered MTM as defined above. When a collectin-based engineered MTM has fewer that all of the domains of a wild-type collectin, the MTM will be a domain-variant engineered MTM or a sequence- and domain-variant engineered MTM as defined above.

Collectin-based engineered MTMs comprise a microbe-binding domain derived from at least one carbohydrate-binding protein selected from the group consisting of: MBL; SP-A; SP-D; CL-L1, CL-P1; CL-34; CL-46; CL-K1, conglutinin; maltose-binding protein; arabinose-binding protein; glucose-binding protein; Galanthus nivalis agglutinin; peanut lectin; lentil lectin; DC-SIGN; and C-reactive protein; and any combinations thereof.

### MBL

In particular aspects and embodiments of the invention, the MTM may be MBL, whether full-length human MBL (SEQ ID NO:1), mature human MBL without the signal sequence (e.g. SEQ ID NO:2), a truncated human MBL that retains microbe surface-binding (e.g. SEQ ID NO:3), the carbohydrate recognition domain (CRD) of human MBL (e.g. SEQ ID NO:4), or the neck and carbohydrate recognition domain of human MBL (e.g. SEQ ID NO:5), whether used alone or in combination with a second protein in the form of a fusion protein, such as a FcMBL protein as defined herein.

The amino acid sequence of full-length human MBL (SEQ ID NO:1; GenBank: AAH69338.1) is:

MSLFPSLPLL LLSMVAASYS ETVTCEDAQK TCPAVIACSS
PGINGFPGKD GRDGTKGEKG EPGQGLRGLQ GPPGKLGPPG
NPGPSGSPGP KGQKGDPGKS PDGDSSLAAS ERKALQTEMA
RIKKWLTFSL GKQVGNKFFL TNGEIMTFEK VKALCVKFQA
SVATPRNAAE NGAIQNLIKE EAFLGITDEK TEGQFVDLTG
NRLTYTNWNE GEPNNAGSDE DCVLLLKNGQ WNDVPCSTSH
LAVCEFPI

The amino acid sequence of mature human MBL without the signal sequence (SEQ ID NO:2) is:

ETVTCEDAQK TCPAVIACSS PGINGFPGKD GRDGTKGEKG
EPGQGLRGLQ GPPGKLGPPG NPGPSGSPGP KGQKGDPGKS
PDGDSSLAAS ERKALQTEMA RIKKWLTFSL GKQVGNKFFL
TNGEIMTFEK VKALCVKFQA SVATPRNAAE NGAIQNLIKE
EAFLGITDEK TEGQFVDLTG NRLTYTNWNE GEPNNAGSDE
DCVLLLKNGQ WNDVPCSTSH LAVCEFPI

The amino acid sequence of a truncated MBL that retains microbe surface-binding (SEQ ID NO:3) is:

AASERKALQT EMARIKKWLT FSLGKQVGNK FFLTNGEIMT
FEKVKALCVK FQASVATPRN AAENGAIQNL IKEEAFLGIT
DEKTEGQFVD LTGNRLTYTN WNEGEPNNAG SDEDCVLLLK
NGQWNDVPCS TSHLAVCEFP I

The amino acid sequence of the carbohydrate recognition domain (CRD) of human MBL (SEQ ID NO:4) is:

VGNKFFLTNG EIMTFEKVKA LCVKFQASVA TPRNAAENGA
IQNLIKEEAF LGITDEKTEG QFVDLTGNRL TYTNWNEGEP
NNAGSDEDCV LLLKNGQWND VPCSTSHLAV CEFPI

The amino acid sequence of the neck and carbohydrate recognition domain of MBL (SEQ ID NO:5) is:

PDGDSSLAAS ERKALQTEMA RIKKWLTFSL GKQVGNKFFL
TNGEIMTFEK VKALCVKFQA SVATPRNAAE NGAIQNLIKE
EAFLGITDEK TEGQFVDLTG NRLTYTNWNE GEPNNAGSDE
DCVLLLKNGQ WNDVPCSTSH LAVCEFPI

The truncated forms of the naturally-occurring protein include portions of any one of SEQ ID NOs:1-5 lacking 1, 2, 3, 4, 5, 6, 7, 8, 9, 10, 15, 20, 25, 30, 35, 40, 45, 50, 55, 60, 65, 70, 75, 80 or more amino acids from the amino-terminus of the protein, or the carboxy-terminus of the protein, or internally within the protein, or any combination thereof.

Alternatively, the truncated forms of the naturally-occurring protein of any one of SEQ ID NOs:1-5 have a deletion of at least 1%, 2%, 3%, 4%, 5%, 6%, 7%, 8%, 9%, 10%, 15%, 20%, 25%, 30%, 35%, 40%, 45% or 50% of the amino acids from the amino-terminus of the protein, or the carboxy-terminus of the protein, or internally within the protein, or any combination thereof.

As to particularly useful truncated forms of the protein, one example is the full-length amino acid sequence of the carbohydrate recognition domain (CRD) of MBL, shown in SEQ ID NO:4. In addition, suitable CRDs include CRDs having an amino acid sequence of about 10 to about 110 amino acid residues, or about 50 to about 100 amino acid residues, of SEQ ID NO:4. In some aspects, the microbe-binding domain can have an amino acid sequence of at least about 5, at least about 10, at least about 15, at least about 20, at least about 30, at least about 40, at least about 50, at least about 60, at least about 70, at least about 80, at least about 90, at least about 100, at least about 110 amino acid residues or more, of SEQ ID NO:4. Accordingly, in some aspects, the carbohydrate recognition domain of an engineered MBL protein can comprise SEQ ID NO:4. In some aspects, the carbohydrate recognition domain of an engineered MBL protein can comprise a fragment of SEQ ID NO:4 as defined above. Also, exemplary amino acid sequences of such fragments include, but are not limited to, ND, EZN (where Z is any amino acid, e.g., P), NEGEPNNAGS (SEQ ID NO:10) or a fragment thereof comprising EPN, GSDEDCVLL or a fragment thereof comprising E, and LLLKNGQWNDVPCST (SEQ ID NO:11) or a fragment thereof comprising ND. Modifications to such CRD fragments, e.g., by conservative substitution (i.e., where an amino acid is replace by an amino acid within the same class of amino acids, where the classes are: aliphatic amino acids (G, A, L, V, I); hydroxyl or sulfur/selenium-containing amino acids (S, C, U, T, M); aromatic amino acids (F, Y, W); basic amino acids (H, K, R); and acidic amino acids (D, E, N, Q)), are also within the scope described herein. In some aspects, the MBL or a fragment thereof used in the microbe surface-binding domain of the engineered MBLs described herein can be a wild-type molecule or a recombinant molecule.

The sequence variants of the naturally-occurring protein and the truncated forms thereof (e.g. SEQ ID NOs:1-5) include proteins having at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% sequence identity to any one of SEQ ID NOs:1-5, or truncated forms thereof, that retain the ability to reduce platelet activation in blood of the protein upon which they are based.

### Engineered MTM Comprising MBL

In other particular aspects and embodiments of the invention, the MTM is an engineered MTM comprising MBL, as defined above, and a second protein in the form of a fusion protein. An exemplary fusion protein comprises some or all of naturally-occurring MBL, such as the carbohydrate recognition domain (CRD) of MBL, and a portion of an immunoglobulin, such as the Fc domain. In use, the Fc domain dimerizes and strengthens the avidity and affinity of the binding by MBLs to monomeric sugars. In some aspects, the N-terminus of fusion proteins can further comprise an oligopeptide linker adapted to bind a solid substrate and orient the CRD of the MBL domain away from a substrate to which it is immobilized. As discussed above, engineered forms of MBLs are known in the art and include each of the forms of MBL disclosed in U.S. Pat. No. 9,150,631, U.S. Patent Pub. 2016/0311877, U.S. Patent Pub. 2019/0077850, U.S. Pat. No. 9,593,160, U.S. Pat. No. 10,435,457, U.S. Patent Pub. 2015/0173883 and International Application Publication No. WO 2011/090954, the entire disclosures of which are hereby incorporated by reference in their entirety.

FcMBL is a specific engineered form of MBL of the invention that comprises the neck and CRD domains of MBL linked to an IgG Fc domain. Proline 81 of mature MBL (SEQ ID NO:2) is a convenient N-terminal point at which to begin the sequence of this engineered construct. For example, the neck and CRD domains (SEQ ID NO:5) of MBL are fused downstream (C-terminal) to the Fc domain of human IgG (Fcy). The Fc domain may include the CH2-CH3 interface of the IgG Fc domain, which contains the binding sites for a number of Fc receptors including Staphylococcal protein A. In use, the Fc domain dimerizes and strengthens the avidity and affinity of the binding by MBLs to monomeric sugars. FcMBL is described in detail in U.S. Pat. No. 9,150,631, the entire disclosure of which is hereby incorporated by reference in its entirety.

Specific examples of FcMBLs that may be used in each of the aspects and embodiments of the invention include, but are not limited to, proteins where the neck and CRD domains of MBL are linked to an Fc component of human IgG1, with examples of the resulting constructs set forth in SEQ ID NOs:6, 7 and 9, and proteins where the CRD domain alone of MBL is linked to an Fc component of human IgG1, with an example of the resulting construct set forth in SEQ ID NO:8.

FcMBL.81 (SEQ ID NO: 6):
EPKSSDKTHT CPPCPAPELL GGPSVFLFPP KPKDTLMISR
TPEVTCVVVD VSHEDPEVKF NWYVDGVEVH NAKTKPREEQ
YNSTYRVVSV LTVLHQDWLN GKEYKCKVSN KALPAPIEKT
ISKAKGQPRE PQVYTLPPSR DELTKNQVSL TCLVKGFYPS
DIAVEWESNG QPENNYKTTP PVLDSDGSFF LYSKLTVDKS
RWQQGNVFSC SVMHEALHNH YTQKSLSLSP GAPDGDSSLA
ASERKALQTE MARIKKWLTF SLGKQVGNKF FLTNGEINIT
FEKVKALCVK FQASVATPRN AAENGAIQNL IKEEAFLGIT
DEKTEGQFVD LTGNRLTYTN WNEGEPNNAG SDEDCVLLLK
NGQWNDVPCS TSHLAVCEFP I
AKT-FcMBL (SEQ ID NO: 7):
AKTEPKSSDK THTCPPCPAP ELLGGPSVFL FPPKPKDTLM
ISRTPEVTCV VVDVSHEDPE VKFNWYVDGV EVHNAKTKPR
EEQYNSTYRV VSVLTVLHQD WLNGKEYKCK VSNKALPAPI
EKTISKAKGQ PREPQVYTLP PSRDELTKNQ VSLTCLVKGF
YPSDIAVEWE SNGQPENNYK TTPPVLDSDG SFFLYSKLTV
DKSRWQQGNV FSCSVMHEAL HNHYTQKSLS LSPGAPDGDS
SLAASERKAL QTEMARIKKW LTFSLGKQVG NKFFLTNGEI
MTFEKVKALC VKFQASVATP RNAAENGAIQ NLIKEEAFLG
ITDEKTEGQF VDLTGNRLTY TNWNEGEPNN AGSDEDCVLL
LKNGQWNDVP CSTSHLAVCE FPI
FcMBL.111 (SEQ ID NO: 8):
EPKSSDKTHT CPPCPAPELL GGPSVFLFPP KPKDTLMISR
TPEVTCVVVD VSHEDPEVKF NWYVDGVEVH NAKTKPREEQ
YNSTYRVVSV LTVLHQDWLN GKEYKCKVSN KALPAPIEKT
ISKAKGQPRE PQVYTLPPSR DELTKNQVSL TCLVKGFYPS
DIAVEWESNG QPENNYKTTP PVLDSDGSFF LYSKLTVDKS
RWQQGNVFSC SVMHEALHNH YTQKSLSLSP GATSKQVGNK
FFLTNGEEVI TFEKVKALCV KFQASVATPR NAAENGAIQN
LIKEEAFLGI TDEKTEGQFV DLTGNRLTYT NWNEGEPNNA
GSDEDCVLLL KNGQWNDVPC STSHLAVCEF PI
FcMBL (SEQ ID NO: 9):
AKTEPKSSDK THTCPPCPAP ELLGGPSVFL FPPKPKDTLM
ISRTPEVTCV VVDVSHEDPE VKFNWYVDGV EVHNAKTKPR
EEQYDSTYRV VSVLTVLHQD WLNGKEYKCK VSNKALPAPI
EKTISKAKGQ PREPQVYTLP PSRDELTKNQ VSLTCLVKGF
YPSDIAVEWE SNGQPENNYK TTPPVLDSDG SFFLYSKLTV
DKSRWQQGNV FSCSVMHEAL HNHYTQKSLS LSPGAPDGDS
SLAASERKAL QTEMARIKKW LTFSLGKQVG NKFFLINGEI
MTFEKVKALC VKFQASVATP RNAAENGAIQ NLIKEEAFLG
ITDEKTEGQF VDLTGNRLTY TNWNEGEPNN AGSDEDCVLL
LKNGQWNDVP CSTSHLAVCE FPI

In SEQ ID NO:9, the residues with a single underscore correspond to the Fc portion, the residues with a double underscore correspond to the MBL neck, and those residues without underscore correspond to the MBL carbohydrate-binding domain.

Various genetically engineered versions of the MTM (e.g., FcMBL) are described in International Application Pub. Nos. WO 2011/090954 and WO 2013/012924, as well as U.S. Pat. Nos. 9,150,631 and 9,593,160, the contents of each of which are incorporated herein by reference in their entireties. Lectins and other mannose binding molecules are also described in, for example, U.S. Pat. Nos. 9,150,631 and 9,632,085, and PCT application publication nos. WO/2011/090954, WO2013012924 and WO/2013/130875, the contents of all of which are incorporated herein by reference in their entireties.

In aspects where the MTM is FcMBL, the Fc region or a fragment thereof can comprise at least one mutation, e.g., to modify the performance of the engineered MBL. For example, in some aspects, the half-life of the engineered MBL described herein can be increased, e.g., by mutating the lysine (K) at the residue 232 to alanine (A) as shown in the Fc domain sequence provided in SEQ ID NO:12. Other mutations, e.g., located at the interface between the CH2 and CH3 domains shown in Hinton et al (2004) J Biol Chem. 279:6213-6216 and Vaccaro C. et al. (2005) Nat Biotechnol. 23: 1283-1288, can be also used to increase the half-life of the IgG1 and thus the engineered MBL.

SEQ ID NO: 12:
EPKSSDKTHT CPPCPAPELL GGPSVFLFPP KPKDTLMISR
TPEVTCVVVD VSHEDPEVKF NWYVDGVEVH NAKTKPREEQ
YNSTYRVVSV LTVLHQDWLN GKEYKCKVSN KALPAPIEKT
ISKAKGQPRE PQVYTLPPSR DELTKNQVSL TCLVKGFYPS
DIAVEWESNG QPENNYKTTP PVLDSDGSFF LYSKLTVDKS
RWQQGNVFSC SVMHEALHNH YTQKSLSLSP GA

In some aspects and embodiments, the FcMBL of the invention comprises or consists of an amino acid sequence having at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% sequence identity to any one of SEQ ID NOs:6-9, that retain the active of protein upon which they are based.

The exemplary MBL sequences provided herein are not construed to be limiting. For example, while the exemplary sequences provided herein are derived from a human species, amino acid sequences of the same carbohydrate recognition domain in plants and other animal species such as mice, rats, porcine, bovine, feline, and canine are known in the art and within the scope described herein.

### Other Collectins

In addition to the aspects and embodiments of the invention defined above that comprise the collectin MBL (whether naturally-occurring or engineered), the present invention encompasses use of any other collectin in an MTM that binds MAMPs, when used in conjunction with the methods and compositions of the invention. Thus, any of the following additional collectins may also be used in the aspects and embodiments of the invention as defined herein: (i) surfactant protein A (SP-A; SEQ ID NO:13), (ii) surfactant protein D (SP-D; SEQ ID NO:14), (iii) collectin liver 1 (CL-Ll; SEQ ID NO:15), (iv) collectin placenta 1 (CL-P1; SEQ ID NO:16), (v) conglutinin collectin of 43 kDa (CL-43; SEQ ID NO:17), (vi) collectin of 46 kDa (CL-46; SEQ ID NO:18), (vii) collectin kidney 1 (CL-K1; SEQ ID NO:19), and (viii) conglutinin (SEQ ID NO:20).

As with MBL, both naturally-occurring collectins and engineered forms of the proteins may be used in the invention. Engineered forms of the proteins include, but are not limited to, truncated forms of the naturally-occurring proteins, sequence variants of the naturally-occurring proteins, sequence variants of the truncated forms of the proteins, fusion proteins comprising the naturally-occurring protein, fusion proteins comprising the truncated forms of the proteins, and fusion proteins comprising the sequence variants.

The truncated forms of the naturally-occurring collectins include portions of any one of SEQ ID NOs:13-20 lacking 1, 2, 3, 4, 5, 6, 7, 8, 9, 10, 15, 20, 25, 30, 35, 40, 45, 50, 55, 60, 65, 70, 75, 80 or more amino acids from the amino-terminus of the protein, or the carboxy-terminus of the protein, or internally within the protein, or any combination thereof.

Alternatively, the truncated forms of the naturally-occurring protein of any one of SEQ ID NOs:13-20 have a deletion of at least 1%, 2%, 3%, 4%, 5%, 6%, 7%, 8%, 9%, 10%, 15%, 20%, 25%, 30%, 35%, 40%, 45% or 50% of the amino acids from the amino-terminus of the protein, or the carboxy-terminus of the protein, or internally within the protein, or any combination thereof.

The sequence variants of the naturally-occurring protein and the truncated forms thereof (e.g. SEQ ID NOs:13-20) include proteins having at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% sequence identity to any one of SEQ ID NOs:13-20, or truncated forms thereof, that retain the activity of the protein upon which they are based.

### Ficolin-Based MTMs

Ficolins are a family of lectins that activate the lectin pathway of complement activation upon binding to a pathogen. Ficolins are soluble molecules comprising pattern recognition receptors (PRRs) within a microbe-binding domain that recognize and selectively bind acetylated compounds, typically N-acetylglucosamine (GlcNAc), produced by pathogens. The lectin pathway is activated by binding of a ficolin to an acetylated compound on the pathogen surface, which activates the serine proteases MASP-1 and MASP-2, which then cleave C4 into C4a and C4b, and cleave C2 into C2a and C2b. C4b and C2b then bind together to form C3-convertase of the classical pathway, leading to the eventual lysis of the target cell via the remainder of the steps in the classical pathway.

Members of the family have a common structure, characterized by three parts or domains arranged in the following N- to C-terminal arrangement: (i) a short N-terminal domain, (ii) a collagen-like domain, and (iii) a fibrinogen-like domain that makes up the microbe-binding domain. The functional form of the molecule is a trimer made up of three identical chains. See FIG. 1.

There are currently three recognized members of the family: ficolin 1 (M-ficolin), ficolin 2 (L-ficolin), and ficolin 3 (H-ficolin). Each of these proteins is an MTM of the invention. The amino acid sequences of the human forms of the proteins are provided in the following paragraphs, with the fibrinogen-like domain underlined:

Ficolin 1 precursor - SEQ ID NO: 21 -
NP_001994.2 - Homo sapiens
MELSGATMAR GLAVLLVLFL HIKNLPAQAA DTCPEVKVVG
LEGSDKLTIL RGCPGLPGAP GPKGEAGVIG ERGERGLPGA
PGKAGPVGPK GDRGEKGMRG EKGDAGQSQS CATGPRNCKD
LLDRGYFLSG WHTIYLPDCR PLTVLCDMDT DGGGWTVFQR
RMDGSVDFYR DWAAYKQGFG SQLGEFWLGN DNIHALTAQG
SSELRVDLVD FEGNHQFAKY KSFKVADEAE KYKLVLGAFV
GGSAGNSLTG HNNNFFSTKD QDNDVSSSNC AEKFQGAWWY
ADCHASNLNG LYLMGPHESY ANGINWSAAK GYKYSYKVSE
MKVRPA
Ficolin 2 isoform a precursor - SEQ ID NO: 22 -
NP_004099.2 - Homo sapiens
MELDRAVGVL GAATLLLSFL GMAWALQAAD TCPEVKMVGL
EGSDKLTILR GCPGLPGAPG PKGEAGTNGK RGERGPPGPP
GKAGPPGPNG APGEPQPCLT GPRTCKDLLD RGHFLSGWHT
IYLPDCRPLT VLCDMDTDGG GWTVFQRRVD GSVDFYRDWA
TYKQGFGSRL GEFWLGNDNI HALTAQGTSE LRVDLVDFED
NYQFAKYRSF KVADEAEKYN LVLGAFVEGS AGDSLTFHNN
QSFSTKDQDN DLNTGNCAVM FQGAWWYKNC HVSNLNGRYL
RGTHGSFANG INWKSGKGYN YSYKVSEMKV RPA
Ficolin 3 isoform 1 precursor - SEQ ID NO: 23 -
NP_003656.2 - Homo sapiens
MDLLWILPSL WLLLLGGPAC LKTQEHPSCP GPRELEASKV
VLLPSCPGAP GSPGEKGAPG PQGPPGPPGK MGPKGEPGDP
VNLLRCQEGP RNCRELLSQG ATLSGWYHLC LPEGRALPVF
CDMDTEGGGW LVFQRRQDGS VDFFRSWSSY RAGFGNQESE
FWLGNENLHQ LTLQGNWELR VELEDFNGNR TFAHYATFRL
LGEVDHYQLA LGKFSEGTAG DSLSLHSGRP FTTYDADHDS
SNSNCAVIVH GAWWYASCYR SNLNGRYAVS EAAAHKYGID
WASGRGVGHP YRRVRMMLR

As with the collectins, both naturally-occurring ficolins and engineered forms of the proteins may be used in the invention. Engineered forms of the proteins include, but are not limited to, truncated forms of the naturally-occurring proteins, sequence variants of the naturally-occurring proteins, sequence variants of the truncated forms of the proteins, fusion proteins comprising the naturally-occurring protein, fusion proteins comprising the truncated forms of the proteins, and fusion proteins comprising the sequence variants.

The truncated forms of the naturally-occurring ficolins include portions of any one of SEQ ID NOs:21-23 lacking 1, 2, 3, 4, 5, 6, 7, 8, 9, 10, 15, 20, 25, 30, 35, 40, 45, 50, 55, 60, 65, 70, 75, 80 or more amino acids from the amino-terminus of the protein, or the carboxy-terminus of the protein, or internally within the protein, or any combination thereof.

Alternatively, the truncated forms of the naturally-occurring protein of any one of SEQ ID NOs:21-23 have a deletion of at least 1%, 2%, 3%, 4%, 5%, 6%, 7%, 8%, 9%, 10%, 15%, 20%, 25%, 30%, 35%, 40%, 45% or 50% of the amino acids from the amino-terminus of the protein, or the carboxy-terminus of the protein, or internally within the protein, or any combination thereof.

The sequence variants of the naturally-occurring protein and the truncated forms thereof (e.g. SEQ ID NOs:21-23) include proteins having at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% sequence identity to any one of SEQ ID NOs:21-23, or truncated forms thereof that retain the activity of the protein upon which they are based.

The MTMs of the invention also include other ficolin-based molecules that bind to one or more MAMPs (acetylated compounds for the ficolins), e.g. those MTMs comprising at least a portion (e.g. domain) of a ficolin-based molecule in the case of an engineered MTM. As used herein, the term “ficolin-based molecule” refers to a molecule comprising a microbe-binding domain derived from a ficolin. The term “ficolin” as used herein refers to any molecule including proteins, natural or genetically modified (e.g., recombinant), that interacts specifically with acetylated compounds (e.g., GlcNAc). The term “ficolin” as used herein can also refer to ficolins derived from any species, including, but not limited to, plants, animals (e.g. mammals, such as human), insects and microorganisms, having the desired binding specificity.

Ficolin-based engineered MTMs of the invention are MTMs that comprise at least a microbe-binding domain of a ficolin, e.g. the fibrinogen-like domain of a ficolin. These MTMs may also include one or more of the other domains of a ficolin, e.g. a short N-terminal domain and/or a collagen-like domain, as well as one or more domains not typically found in a ficolin, such as a collectin cysteine-rich domain, a collectin collagen-like domain, a collectin coiled-coil neck domain, a TLR transmembrane helix, a TLR C-terminal cytoplasmic signaling domain, an oligomerization domain, a signal domain, an anchor domain, a collagen-like domain, a fibrinogen-like domain, an immunoglobulin domain, and an immunoglobulin-like domain. When a ficolin-based engineered MTM has each of the domains of a wild-type ficolin, the MTM will be a sequence-variant engineered MTM as defined above. When a ficolin-based engineered MTM has fewer that all of the domains of a wild-type ficolin, the MTM will be a domain-variant engineered MTM or a sequence- and domain-variant engineered MTM as defined above.

Ficolin-based engineered MTMs comprise a microbe-binding domain comprising at least one fibrinogen-like domain of a ficolin selected from the group consisting of ficolin 1, ficolin 2, and ficolin 3.

In certain aspects, the at least one additional domain is an immunoglobulin domain. For example, the immunoglobulin domain may comprise the amino acid sequence of SEQ ID NO:12 or a sequence variant thereof having at least 85% sequence identity to SEQ ID NO:12.

In some aspects, the MTMs and engineered MTMs of the invention comprise a microbe-binding domain comprising the fibrinogen-like domain of ficolin 1 of SEQ ID NO:21, optionally with an immunoglobulin domain of SEQ ID NO:12. In other aspects, the MTMs and engineered MTMs of the invention comprise a microbe-binding domain comprising the fibrinogen-like domain of ficolin 2 of SEQ ID NO:22, optionally with an immunoglobulin domain of SEQ ID NO:12. In further aspects, the MTMs and engineered MTMs of a microbe-binding domain comprising the fibrinogen-like domain of ficolin 3 of SEQ ID NO:23, optionally with an immunoglobulin domain of SEQ ID NO:12.

In some aspects, the engineered MTMs of the invention comprise an microbe-binding domain having an amino acid sequence selected from SEQ ID NO:21-SEQ ID NO:23, or an amino acid sequence that is at least 80, 81, 82, 83, 84, 85, 86, 87, 88, 89, 90, 91, 92, 93, 94, 95, 96, 97, 98, or 99% identical to any one of SEQ ID NO:21-SEQ ID NO:23, but less than 100% identical, and that retains the microbe-binding activity of the wild-type protein.

In some aspects, the ficolin-based engineered MTMs comprise a ficolin microbe-binding domain comprising the fibrinogen-like domain of any one of SEQ ID NOs:21, 22 and 23 or a sequence variant thereof having at least 85% sequence identity to any one of SEQ ID NOs:21, 22 and 23 and an immunoglobulin domain comprising the amino acid sequence of SEQ ID NO:12 or a sequence variant thereof having at least 85% sequence identity to SEQ ID NO:12.

In some aspects, the microbe-binding domain comprising a fibrinogen-like domain of a ficolin from a primate, mouse, rat, hamster, rabbit, or any other species as described herein.

The exemplary sequences provided herein for the ficolins are not to be construed as limiting. For example, while the exemplary sequences provided herein are derived from a human, amino acid sequences of ficolins from other species such as mice, rats, porcine, bovine, feline, and canine are known in the art and within the scope described herein.

### Toll-Like Receptor-Based MTMs

Toll-like receptors (TLRs) comprise a family of proteins that are integral to the proper functioning of the innate immune system. The proteins are type I integral membrane proteins (i.e. single-pass, membrane-spanning receptors) that are typically found on the surface of sentinel cells, such as macrophages and dendritic cells, but can also be found on the surface of other leukocytes including natural killer cells, T cells and B cells, and non-immune cells including epithelial cell, endothelial cells, and fibroblasts. After microbes have gained entry to a subject, such as a human, through the skin or mucosa, they are recognized by TLR-expressing cells, which leads to innate immune responses and the development of antigen-specific acquired immunity. TLRs thus recognize MAMPs by microbes.

Members of the family have a common structure, characterized by three parts or domains arranged in the following N- to C-terminal arrangement: (i) an N-terminal ligand-binding domain, i.e. the microbe-binding domain, (ii) a single transmembrane helix (˜20 amino acids), and (iii) a C-terminal cytoplasmic signaling domain.

The ligand-binding domain is a glycoprotein comprising 550-800 amino acid residues (depending on the identity of the TLR), constructed of tandem copies of leucine-rich repeats (LRR), which are typically 22-29 residues in length and that contains hydrophobic residues spaced at distinctive intervals. The receptors share a common structural framework in their extracellular, ligand-binding domains. The domains each adopt a horseshoe-shaped structure formed by the leucine-rich repeat motifs.

The functional form of a TLR is a dimer, with both homodimers and heterodimers being known. In the case of heterodimers, the different TLRs in the dimer may have different ligand specificities. Upon ligand binding, TLRs dimerize their ectodomains via their lateral faces, forming “m”-shaped structures. Dimerization leads to downstream signaling.

A set of endosomal TLRs comprising TLR3, TLR7, TLR8 and TLR9 recognize nucleic acids derived from viruses as well as endogenous nucleic acids in context of pathogenic events. Activation of these receptor leads to production of inflammatory cytokines as well as type I interferons (interferon type I) to help fight viral infection.

There are a number of recognized human members of the family, including TLR1, TLR2, TLR3, TLR4, TLR5, TLR6, TLR7, TLR8, TLR9, and TLR10. Each of these proteins may be used as an MTM of the invention. The amino acid sequences of the human forms of the proteins are provided in the following paragraphs, with the extracellular domain that comprises the N-terminal ligand-binding domain underlined:

TLR1 (toll-like receptor 1 precursor) - NCBI Reference Sequence: NP_003254.2
(SEQ ID NO: 24)
MTSIFHFAII FMLILQIRIQ LSEESEFLVD RSKNGLIHVP KDLSQKTTIL NISQNYISEL
WTSDILSLSK LRILIISHNR IQYLDISVFK FNQELEYLDL SHNKLVKISC HPTVNLKHLD
LSFNAFDALP ICKEFGNMSQ LKFLGLSTTH LEKSSVLPIA HLNISKVLLV LGETYGEKED
PEGLQDFNTE SLHIVFPINK EFHFILDVSV KTVANLELSN IKCVLEDNKC SYFLSILAKL
QTNPKLSNLT LNNIETTWNS FIRILQLVWH TTVWYFSISN VKLQGQLDFR DFDYSGTSLK
ALSIHQVVSD VFGFPQSYIY EIFSNMNIKN FTVSGTRMVH MLCPSKISPF LHLDFSNNLL
TDTVFENCGH LTELETLILQ MNQLKELSKI AEMTTQMKSL QQLDISQNSV SYDEKKGDCS
WTKSLLSLNM SSNILTDTIF RCLPPRIKVL DLHSNKIKSI PKQVVKLEAL QELNVAFNSL
TDLPGCGSFS SLSVLIIDHN SVSHPSADFF QSCQKMRSIK AGDNPFQCTC ELGEFVKNID
QVSSEVLEGW PDSYKCDYPE SYRGTLLKDF HMSELSCNIT LLIVTIVATM LVLAVTVTSL
CSYLDLPWYL RMVCQWTQTR RRARNIPLEE LQRNLQFHAF ISYSGHDSFW VKNELLPNLE
KEGMQICLHE RNFVPGKSIV ENIITCIEKS YKSIFVLSPN FVQSEWCHYE LYFAHHNLFH
EGSNSLILIL LEPIPQYSIP SSYHKLKSLM ARRTYLEWPK EKSKRGLFWA NLRAAINIKL
TEQAKK
TLR2 (toll-like receptor 2 precursor) - NCBI Reference Sequence:
NP_001305722.1
(SEQ ID NO: 25)
MPHTLWMVWV LGVIISLSKE ESSNQASLSC DRNGICKGSS GSLNSIPSGL TEAVKSLDLS
NNRITYISNS DLQRCVNLQA LVLTSNGINT IEEDSFSSLG SLEHLDLSYN YLSNLSSSWF
KPLSSLTFLN LLGNPYKTLG ETSLFSHLTK LQILRVGNMD TFTKIQRKDF AGLTFLEELE
IDASDLQSYE PKSLKSIQNV SHLILHMKQH ILLLEIFVDV TSSVECLELR DTDLDTFHFS
ELSTGETNSL IKKFTFRNVK ITDESLFQVM KLLNQISGLL ELEFDDCTLN GVGNFRASDN
DRVIDPGKVE TLTIRRLHIP RFYLFYDLST LYSLTERVKR ITVENSKVFL VPCLLSQHLK
SLEYLDLSEN LMVEEYLKNS ACEDAWPSLQ TLILRQNHLA SLEKTGETLL TLKNLTNIDI
SKNSFHSMPE TCQWPEKMKY LNLSSTRIHS VTGCIPKTLE ILDVSNNNLN LFSLNLPQLK
ELYISRNKLM TLPDASLLPM LLVLKISRNA ITTFSKEQLD SFHTLKTLEA GGNNFICSCE
FLSFTQEQQA LAKVLIDWPA NYLCDSPSHV RGQQVQDVRL SVSECHRTAL VSGMCCALFL
LILLTGVLCH RFHGLWYMKM MWAWLQAKRK PRKAPSRNIC YDAFVSYSER DAYWVENLMV
QELENFNPPF KLCLHKRDFI PGKWIIDNII DSIEKSHKTV FVLSENFVKS EWCKYELDFS
HFRLFDENND AAILILLEPI EKKAIPQRFC KLRKIMNTKT YLEWPMDEAQ REGFWVNLRA
AIKS
TLR3 (toll-like receptor 3 precursor) - NCBI Reference Sequence: NP_003256.1
(SEQ ID NO: 26)
MRQTLPCIYF WGGLLPFGML CASSTTKCTV SHEVADCSHL KLTQVPDDLP TNITVLNLTH
NQLRRLPAAN FTRYSQLTSL DVGFNTISKL EPELCQKLPM LKVLNLQHNE LSQLSDKTFA
FCTNLTELHL MSNSIQKIKN NPFVKQKNLI TLDLSHNGLS STKLGTQVQL ENLQELLLSN
NKIQALKSEE LDIFANSSLK KLELSSNQIK EFSPGCFHAI GRLFGLFLNN VQLGPSLTEK
LCLELANTSI RNLSLSNSQL STTSNTTFLG LKWTNLTMLD LSYNNLNVVG NDSFAWLPQL
EYFFLEYNNI QHLFSHSLHG LFNVRYLNLK RSFTKQSISL ASLPKIDDFS FQWLKCLEHL
NMEDNDIPGI KSNMFTGLIN LKYLSLSNSF TSLRTLTNET FVSLAHSPLH ILNLTKNKIS
KIESDAFSWL GHLEVLDLGL NEIGQELTGQ EWRGLENIFE IYLSYNKYLQ LTRNSFALVP
SLQRLMLRRV ALKNVDSSPS PFQPLRNLTI LDLSNNNIAN INDDMLEGLE KLEILDLQHN
NLARLWKHAN PGGPIYFLKG LSHLHILNLE SNGFDEIPVE VFKDLFELKI IDLGLNNLNT
LPASVFNNQV SLKSLNLQKN LITSVEKKVF GPAFRNLTEL DMRFNPFDCT CESIAWFVNW
INETHTNIPE LSSHYLCNTP PHYHGFPVRL FDTSSCKDSA PFELFFMINT SILLIFIFIV
LLIHFEGWRI SFYWNVSVHR VLGFKEIDRQ TEQFEYAAYI IHAYKDKDWV WEHFSSMEKE
DQSLKFCLEE RDFEAGVFEL EAIVNSIKRS RKIIFVITHH LLKDPLCKRF KVHHAVQQAI
EQNLDSIILV FLEEIPDYKL NHALCLRRGM FKSHCILNWP VQKERIGAFR HKLQVALGSK
NSVH
TLR4 (toll-like receptor 4 isoform D) - NCBI Reference Sequence: NP_612567.1
(SEQ ID NO: 27)
MMSASRLAGT LIPAMAFLSC VRPESWEPCV EVVPNITYQC MELNFYKIPD NLPFSTKNLD
LSFNPLRHLG SYSFFSFPEL QVLDLSRCEI QTIEDGAYQS LSHLSTLILT GNPIQSLALG
AFSGLSSLQK LVAVETNLAS LENFPIGHLK TLKELNVAHN LIQSFKLPEY FSNLTNLEHL
DLSSNKIQSI YCTDLRVLHQ MPLLNLSLDL SLNPMNFIQP GAFKEIRLHK LTLRNNFDSL
NVMKTCIQGL AGLEVHRLVL GEFRNEGNLE KFDKSALEGL CNLTIEEFRL AYLDYYLDDI
IDLFNCLTNV SSFSLVSVTI ERVKDFSYNF GWQHLELVNC KFGQFPTLKL KSLKRLTFTS
NKGGNAFSEV DLPSLEFLDL SRNGLSFKGC CSQSDFGTTS LKYLDLSFNG VITMSSNFLG
LEQLEHLDFQ HSNLKQMSEF SVFLSLRNLI YLDISHTHTR VAFNGIFNGL SSLEVLKMAG
NSFQENFLPD IFTELRNLTF LDLSQCQLEQ LSPTAFNSLS SLQVLNMSHN NFFSLDTFPY
KCLNSLQVLD YSLNHIMTSK KQELQHFPSS LAFLNLTQND FACTCEHQSF LQWIKDQRQL
LVEVERMECA TPSDKQGMPV LSLNITCQMN KTIIGVSVLS VLVVSVVAVL VYKFYFHLML
LAGCIKYGRG ENIYDAFVIY SSQDEDWVRN ELVKNLEEGV PPFQLCLHYR DFIPGVAIAA
NIIHEGFHKS RKVIVVVSQH FIQSRWCIFE YEIAQTWQFL SSRAGIIFIV LQKVEKTLLR
QQVELYRLLS RNTYLEWEDS VLGRHIFWRR LRKALLDGKS WNPEGTVGTG CNWQEATSI
TLR5 (toll-like receptor 5 precursor) - NCBI Reference Sequence: NP_003259.2
(SEQ ID NO: 28)
MGDHLDLLLG VVLMAGPVFG IPSCSFDGRI AFYRFCNLTQ VPQVLNTTER LLLSFNYIRT
VTASSFPFLE QLQLLELGSQ YTPLTIDKEA FRNLPNLRIL DLGSSKIYFL HPDAFQGLFH
LFELRLYFCG LSDAVLKDGY FRNLKALTRL DLSKNQIRSL YLHPSFGKLN SLKSIDFSSN
QIFLVCEHEL EPLQGKTLSF FSLAANSLYS RVSVDWGKCM NPFRNMVLEI LDVSGNGWTV
DITGNFSNAI SKSQAFSLIL AHHIMGAGFG FHNIKDPDQN TFAGLARSSV RHLDLSHGFV
FSLNSRVFET LKDLKVLNLA YNKINKIADE AFYGLDNLQV LNLSYNLLGE LYSSNFYGLP
KVAYIDLQKN HIAIIQDQTF KFLEKLQTLD LRDNALTTIH FIPSIPDIFL SGNKLVTLPK
INLTANLIHL SENRLENLDI LYFLLRVPHL QILILNQNRF SSCSGDQTPS ENPSLEQLFL
GENMLQLAWE TELCWDVFEG LSHLQVLYLN HNYLNSLPPG VFSHLTALRG LSLNSNRLTV
LSHNDLPANL EILDISRNQL LAPNPDVFVS LSVLDITHNK FICECELSTF INWLNHTNVT
IAGPPADIYC VYPDSFSGVS LESLSTEGCD EEEVLKSLKF SLFIVCTVTL TLFLMTILTV
TKFRGFCFIC YKTAQRLVFK DHPQGTEPDM YKYDAYLCFS SKDFTWVQNA LLKHLDTQYS
DQNRFNLCFE ERDFVPGENR IANIQDAIWN SRKIVCLVSR HFLRDGWCLE AFSYAQGRCL
SDLNSALIMV VVGSLSQYQL MKHQSIRGFV QKQQYLRWPE DLQDVGWFLH KLSQQILKKE
KEKKKDNNIP LQTVATIS
TLR6 (toll-like receptor 6 precursor) - NCBI Reference Sequence: NP_006059.2
(SEQ ID NO: 29)
MTKDKEPIVK SFHFVCLMII IVGTRIQFSD GNEFAVDKSK RGLIHVPKDL PLKTKVLDMS
QNYIAELQVS DMSFLSELTV LRLSHNRIQL LDLSVFKFNQ DLEYLDLSHN QLQKISCHPI
VSFRHLDLSF NDFKALPICK EFGNLSQLNF LGLSAMKLQK LDLLPIAHLH LSYILLDLRN
YYIKENETES LQILNAKTLH LVFHPTSLFA IQVNISVNTL GCLQLTNIKL NDDNCQVFIK
FLSELTRGST LLNFTLNHIE TTWKCLVRVF QFLWPKPVEY LNIYNLTIIE SIREEDFTYS
KTTLKALTIE HITNQVFLFS QTALYTVFSE MNIMMLTISD TPFIHMLCPH APSTFKFLNF
TQNVFTDSIF EKCSTLVKLE TLILQKNGLK DLFKVGLMTK DMPSLEILDV SWNSLESGRH
KENCTWVESI VVLNLSSNML TDSVFRCLPP RIKVLDLHSN KIKSVPKQVV KLEALQELNV
AFNSLTDLPG CGSFSSLSVL IIDHNSVSHP SADFFQSCQK MRSIKAGDNP FQCTCELREF
VKNIDQVSSE VLEGWPDSYK CDYPESYRGS PLKDFHMSEL SCNITLLIVT IGATMLVLAV
TVTSLCIYLD LPWYLRMVCQ WTQTRRRARN IPLEELQRNL QFHAFISYSE HDSAWVKSEL
VPYLEKEDIQ ICLHERNFVP GKSIVENIIN CIEKSYKSIF VLSPNFVQSE WCHYELYFAH
HNLFHEGSNN LILILLEPIP QNSIPNKYHK LKALMTQRTY LQWPKEKSKR GLFWANIRAA
FNMKLTLVTE NNDVKS
TLR7 (toll-like receptor 7 precursor) - NCBI Reference Sequence: NP_057646.1
(SEQ ID NO: 30)
MVFPMWTLKR QILILFNIIL ISKLLGARWF PKTLPCDVTL DVPKNHVIVD CTDKHLTEIP
GGIPTNTTNL TLTINHIPDI SPASFHRLDH LVEIDFRCNC VPIPLGSKNN MCIKRLQIKP
RSFSGLTYLK SLYLDGNQLL EIPQGLPPSL QLLSLEANNI FSIRKENLTE LANIEILYLG
QNCYYRNPCY VSYSIEKDAF LNLTKLKVLS LKDNNVTAVP TVLPSTLTEL YLYNNMIAKI
QEDDFNNLNQ LQILDLSGNC PRCYNAPFPC APCKNNSPLQ IPVNAFDALT ELKVLRLHSN
SLQHVPPRWF KNINKLQELD LSQNFLAKEI GDAKFLHFLP SLIQLDLSFN FELQVYRASM
NLSQAFSSLK SLKILRIRGY VFKELKSFNL SPLHNLQNLE VLDLGTNFIK IANLSMFKQF
KRLKVIDLSV NKISPSGDSS EVGFCSNART SVESYEPQVL EQLHYFRYDK YARSCRFKNK
EASFMSVNES CYKYGQTLDL SKNSIFFVKS SDFQHLSFLK CLNLSGNLIS QTLNGSEFQP
LAELRYLDFS NNRLDLLHST AFEELHKLEV LDISSNSHYF QSEGITHMLN FTKNLKVLQK
LMMNDNDISS STSRTMESES LRTLEFRGNH LDVLWREGDN RYLQLFKNLL KLEELDISKN
SLSFLPSGVF DGMPPNLKNL SLAKNGLKSF SWKKLQCLKN LETLDLSHNQ LTTVPERLSN
CSRSLKNLIL KNNQIRSLTK YFLQDAFQLR YLDLSSNKIQ MIQKTSFPEN VLNNLKMLLL
HHNRFLCTCD AVWFVWWVNH TEVTIPYLAT DVTCVGPGAH KGQSVISLDL YTCELDLTNL
ILFSLSISVS LFLMVMMTAS HLYFWDVWYI YHFCKAKIKG YQRLISPDCC YDAFIVYDTK
DPAVTEWVLA ELVAKLEDPR EKHFNLCLEE RDWLPGQPVL ENLSQSIQLS KKTVFVMTDK
YAKTENFKIA FYLSHQRLMD EKVDVIILIF LEKPFQKSKF LQLRKRLCGS SVLEWPTNPQ
AHPYFWQCLK NALATDNHVA YSQVEKETV
TLR8 (toll-like receptor 8 isoform 1) - UniProtKB/Swiss-Prot: Q9NR97.1
(SEQ ID NO: 31)
MENMFLQSSM LTCIFLLISG SCELCAEENF SRSYPCDEKK QNDSVIAECS NRRLQEVPQT
VGKYVTELDL SDNFITHITN ESFQGLQNLT KINLNHNPNV QHQNGNPGIQ SNGLNITDGA
FLNLKNLREL LLEDNQLPQI PSGLPESLTE LSLIQNNIYN ITKEGISRLI NLKNLYLAWN
CYFNKVCEKT NIEDGVFETL TNLELLSLSF NSLSHVPPKL PSSLRKLFLS NTQIKYISEE
DFKGLINLTL LDLSGNCPRC FNAPFPCVPC DGGASINIDR FAFQNLTQLR YLNLSSTSLR
KINAAWFKNM PHLKVLDLEF NYLVGEIASG AFLTMLPRLE ILDLSFNYIK GSYPQHINIS
RNFSKLLSLR ALHLRGYVFQ ELREDDFQPL MQLPNLSTIN LGINFIKQID FKLFQNFSNL
EIIYLSENRI SPLVKDTRQS YANSSSFQRH IRKRRSTDFE FDPHSNFYHF TRPLIKPQCA
AYGKALDLSL NSIFFIGPNQ FENLPDIACL NLSANSNAQV LSGTEFSAIP HVKYLDLTNN
RLDFDNASAL TELSDLEVLD LSYNSHYFRI AGVTHHLEFI QNFTNLKVLN LSHNNIYTLT
DKYNLESKSL VELVFSGNRL DILWNDDDNR YISIFKGLKN LTRLDLSLNR LKHIPNEAFL
NLPASLTELH INDNMLKFFN WTLLQQFPRL ELLDLRGNKL LFLTDSLSDF TSSLRTLLLS
HNRISHLPSG FLSEVSSLKH LDLSSNLLKT INKSALETKT TTKLSMLELH GNPFECTCDI
GDFRRWMDEH LNVKIPRLVD VICASPGDQR GKSIVSLELT TCVSDVTAVI LFFFTFFITT
MVMLAALAHH LFYWDVWFIY NVCLAKVKGY RSLSTSQTFY DAYISYDTKD ASVTDWVINE
LRYHLEESRD KNVLLCLEER DWDPGLAIID NLMQSINQSK KTVFVLTKKY AKSWNFKTAF
YLALQRLMDE NMDVIIFILL EPVLQHSQYL RLRQRICKSS ILQWPDNPKA EGLFWQTLRN
VVLTENDSRY NNMYVDSIKQ Y
TLR9 (toll-like receptor 9 precursor) - NCBI Reference Sequence: NP_059138.1
(SEQ ID NO: 32)
MGFCRSALHP LSLLVQAIML AMTLALGTLP AFLPCELQPH GLVNCNWLFL KSVPHFSMAA
PRGNVTSLSL SSNRIHHLHD SDFAHLPSLR HLNLKWNCPP VGLSPMHFPC HMTIEPSTFL
AVPTLEELNL SYNNIMTVPA LPKSLISLSL SHTNILMLDS ASLAGLHALR FLFMDGNCYY
KNPCRQALEV APGALLGLGN LTHLSLKYNN LTVVPRNLPS SLEYLLLSYN RIVKLAPEDL
ANLTALRVLD VGGNCRRCDH APNPCMECPR HFPQLHPDTF SHLSRLEGLV LKDSSLSWLN
ASWFRGLGNL RVLDLSENFL YKCITKTKAF QGLTQLRKLN LSFNYQKRVS FAHLSLAPSF
GSLVALKELD MHGIFFRSLD ETTLRPLARL PMLQTLRLQM NFINQAQLGI FRAFPGLRYV
DLSDNRISGA SELTATMGEA DGGEKVWLQP GDLAPAPVDT PSSEDFRPNC STLNFTLDLS
RNNLVTVQPE MFAQLSHLQC LRISHNCISQ AVNGSQFLPL TGLQVLDLSH NKLDLYHEHS
FTELPRLEAL DLSYNSQPFG MQGVGHNFSF VAHLRTLRHL SLAHNNIHSQ VSQQLCSTSL
RALDESGNAL GHMWAEGDLY LHFFQGLSGL IWLDLSQNRL HTLLPQTLRN LPKSLQVLRL
RDNYLAFFKW WSLHFLPKLE VLDLAGNQLK ALTNGSLPAG TRLRRLDVSC NSISFVAPGF
FSKAKELREL NLSANALKTV DHSWFGPLAS ALQILDVSAN PLHCACGAAF MDFLLEVQAA
VPGLPSRVKC GSPGQLQGLS IFAQDLRLCL DEALSWDCFA LSLLAVALGL GVPMLHHLCG
WDLWYCFHLC LAWLPWRGRQ SGRDEDALPY DAFVVFDKTQ SAVADWVYNE LRGQLEECRG
RWALRLCLEE RDWLPGKTLF ENLWASVYGS RKTLFVLAHT DRVSGLLRAS FLLAQQRLLE
DRKDVVVLVI LSPDGRRSRY VRLRQRLCRQ SVLLWPHQPS GQRSFWAQLG MALTRDNHHF
YNRNFCQGPT AE
TLR10 (toll-like receptor 10 isoform a) - NCBI Reference Sequence:
NP_001017388.1
(SEQ ID NO: 33)
MRLIRNIYIF CSIVMTAEGD APELPEEREL MTNCSNMSLR KVPADLTPAT TTLDLSYNLL
FQLQSSDFHS VSKLRVLILC HNRIQQLDLK TFEFNKELRY LDLSNNRLKS VTWYLLAGLR
YLDLSFNDFD TMPICEEAGN MSHLEILGLS GAKIQKSDFQ KIAHLHLNTV FLGFRTLPHY
EEGSLPILNT TKLHIVLPMD TNFWVLLRDG IKTSKILEMT NIDGKSQFVS YEMQRNLSLE
NAKTSVLLLN KVDLLWDDLF LILQFVWHTS VEHFQIRNVT FGGKAYLDHN SFDYSNTVMR
TIKLEHVHFR VFYIQQDKIY LLLTKMDIEN LTISNAQMPH MLFPNYPTKF QYLNFANNIL
TDELFKRTIQ LPHLKTLILN GNKLETLSLV SCFANNTPLE HLDLSQNLLQ HKNDENCSWP
ETVVNMNLSY NKLSDSVFRC LPKSIQILDL NNNQIQTVPK ETIHLMALRE LNIAFNFLTD
LPGCSHFSRL SVLNIEMNFI LSPSLDFVQS CQEVKTLNAG RNPFRCTCEL KNFIQLETYS
EVMMVGWSDS YTCEYPLNLR GTRLKDVHLH ELSCNTALLI VTIVVIMLVL GLAVAFCCLH
FDLPWYLRML GQCTQTWHRV RKTTQEQLKR NVRFHAFISY SEHDSLWVKN ELIPNLEKED
GSILICLYES YFDPGKSISE NIVSFIEKSY KSIFVLSPNF VQNEWCHYEF YFAHHNLFHE
NSDHIILILL EPIPFYCIPT RYHKLKALLE KKAYLEWPKD RRKCGLFWAN LRAAINVNVL
ATREMYELQT FTELNEESRG STISLMRTDC L

As with the collectins, both naturally-occurring TLR and engineered forms of the proteins may be used in the invention. Engineered forms of the proteins include, but are not limited to, truncated forms of the naturally-occurring proteins, sequence variants of the naturally-occurring proteins, sequence variants of the truncated forms of the proteins, fusion proteins comprising the naturally-occurring protein, fusion proteins comprising the truncated forms of the proteins, and fusion proteins comprising the sequence variants.

The truncated forms of the naturally-occurring TLRs include portions of any one of SEQ ID NOs:24-33 lacking 1, 2, 3, 4, 5, 6, 7, 8, 9, 10, 15, 20, 25, 30, 35, 40, 45, 50, 55, 60, 65, 75, 80 or more amino acids from the amino-terminus of the protein, or the carboxy-terminus of the protein, or internally within the protein, or any combination thereof.

Alternatively, the truncated forms of the naturally-occurring protein of any one of SEQ ID NOs:24-33 have a deletion of at least 1%, 2%, 3%, 4%, 5%, 6%, 7%, 8%, 9%, 10%, 15%, 20%, 25%, 30%, 35%, 40%, 45% or 50% of the amino acids from the amino-terminus of the protein, or the carboxy-terminus of the protein, or internally within the protein, or any combination thereof.

The sequence variants of the naturally-occurring protein and the truncated forms thereof (e.g. SEQ ID NOs:24-33) include proteins having at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% sequence identity to any one of SEQ ID NOs:24-33, or truncated forms thereof that retain the activity of the protein upon which they are based.

The MTMs of the invention also include other TLR-based molecules that bind to one or more MAMPs, e.g. those MTMs comprising at least a portion (e.g. domain) of a TLR-based molecule in the case of an engineered MTM. As used herein, the term “TLR-based molecule” refers to a molecule comprising a microbe-binding domain (i.e. an N-terminal ligand-binding domain) derived from a TLR. The term “TLR” as used herein refers to any molecule including proteins, natural or genetically modified (e.g., recombinant), that interacts specifically with an MAMP and that has a Toll IL-1 receptor (TIR) domain in their signaling domain. The term “TLR” as used herein can also refer to TLR derived from any species, including, but not limited to, plants, animals (e.g. mammals, such as human), insects and microorganisms, having the desired binding specificity.

TLR-based engineered MTMs of the invention are MTMs that comprise at least a microbe-binding domain of a TLR, e.g. the N-terminal ligand-binding domain of a TLR. These MTMs may also include one or more of the other domains of a TLR, e.g. a transmembrane helix and/or a C-terminal cytoplasmic signaling domain, as well as one or more domains not typically found in a TLR, such as a ficolin short N-terminal domain, a ficolin collagen-like domain, a collectin cysteine-rich domain, a collectin collagen-like domain, a collectin coiled-coil neck domain, an oligomerization domain, a signal domain, an anchor domain, a collagen-like domain, a fibrinogen-like domain, an immunoglobulin domain, and an immunoglobulin-like domain. When a TLR-based engineered MTM has each of the domains of a wild-type TLR, the MTM will be a sequence-variant engineered MTM as defined above. When a TLR-based engineered MTM has fewer that all of the domains of a wild-type TLR, the MTM will be a domain-variant engineered MTM or a sequence- and domain-variant engineered MTM as defined above.

TLR-based engineered MTMs comprise a microbe-binding domain comprising at least one N-terminal ligand-binding domain of a TLR selected from the group consisting of TLR1, TLR2, TLR3, TLR4, TLR5, TLR6, TLR7, TLR8, TLR9, and TLR10.

In certain aspects of this embodiment, the at least one additional domain is an immunoglobulin domain. For example, the immunoglobulin domain may comprise the amino acid sequence of SEQ ID NO:12 or a sequence variant thereof having at least 85% sequence identity to SEQ ID NO:12.

In some aspects, the MTMs and engineered MTMs of the invention comprise a microbe-binding domain comprising the N-terminal ligand-binding domain of TLR1 of SEQ ID NO:24, or the N-terminal ligand-binding domain of TLR2 of SEQ ID NO:25, or the N-terminal ligand-binding domain of TLR3 of SEQ ID NO:26, or the N-terminal ligand-binding domain of TLR4 of SEQ ID NO:27, or the N-terminal ligand-binding domain of TLR5 of SEQ ID NO:28, or the N-terminal ligand-binding domain of TLR6 of SEQ ID NO:29, or the N-terminal ligand-binding domain of TLR7 of SEQ ID NO:30, or the N-terminal ligand-binding domain of TLR8 of SEQ ID NO:31, or the N-terminal ligand-binding domain of TLR9 of SEQ ID NO:32, or the N-terminal ligand-binding domain of TLR10 of SEQ ID NO:33. In each of these examples, MTMs and engineered MTMs may further comprise an immunoglobulin domain of SEQ ID NO:12.

In some aspects, the engineered MTMs of the invention comprise an microbe-binding domain having an amino acid sequence selected from SEQ ID NO:24-SEQ ID NO:33, or an amino acid sequence that is at least 80, 81, 82, 83, 84, 85, 86, 87, 88, 89, 90, 91, 92, 93, 94, 95, 96, 97, 98, or 99% identical to any one of SEQ ID NO:24-SEQ ID NO:33, but less than 100% identical, and that retains the microbe-binding activity of the wild-type protein.

In certain aspects of this embodiment, the TLR-based engineered MTMs comprise a TLR microbe-binding domain comprising the N-terminal ligand-binding domain of any one of SEQ ID NOs:24-33 or a sequence variant thereof having at least 85% sequence identity to any one of SEQ ID NOs:24-33 and an immunoglobulin domain comprising the amino acid sequence of SEQ ID NO:12 or a sequence variant thereof having at least 85% sequence identity to SEQ ID NO:12.

In some aspects, the microbe-binding domain comprising a N-terminal ligand-binding domain of a TLR from a primate, mouse, rat, hamster, rabbit, or any other subject as described herein.

The exemplary sequences provided herein for the TLRs are not to be construed as limiting. For example, while the exemplary sequences provided herein are derived from a human, amino acid sequences of TLRs from other species such as mice, rats, porcine, bovine, feline, and canine are known in the art and within the scope described herein.

In some further aspects, the MTMs of the invention are those described in at least one of the following: U.S. provisional application numbers 61/296,222, 61/508,957, 61/604,878, 61/605,052, 61/605,081, 61/788,570, 61/846,438, 61/866,843, 61/917,705, 62/201,745, 62/336,940, 62/543,614; PCT application numbers PCT/US2011/021603, PCT/US2012/047201, PCT/US2013/028409 , PCT/US2014/028683, PCT/US2014/046716, PCT/US2014/071293, PCT/US2016/045509, PCT/US2017/032928; U.S. patent application Ser. No. 13/574,191, 14/233,553, 14/382,043, 14/766,575, 14/831,480, 14/904,583, 15/105,298, 15/415,352, 15/483,216, 15/668,794, 15/750,788, 15/839,352, 16/059,799, 16/302,023, 16/553,635; and U.S. Pat. Nos. 9,150,631, 9,593,160, 9,632,085, 9,791,440, and 10,435,457; the contents of each of which are incorporated by reference herein in their entireties.

### Compositions Comprising MTMs

The invention includes compositions comprising one or more of types of MTMs defined herein, i.e. both naturally-occurring MTMs and engineered MTMs. As indicated above, particular MTMs can be defined based on (i) structural terms (e.g. based on the components of the MTM; the amino acid sequence of the MTM; the nucleic acid sequence of the MTM; etc.), (ii) functional terms (e.g. the identity of the MAMP bound by the PRR portion of the microbe-binding domain; the affinity or avidity of binding to the MAMP; etc.), or (iii) both structural and functional terms. When a composition is defined as comprising two or more types of MTMs, it should be understood that “types” of MTMs in the composition differ based on structural and/or functional terms from each other. When there is more than one type of MTM in a composition, the composition is said to comprise a mixture of different types of MTMs within the composition.

An advantage of the present invention is the composition can be customized based on the particular use of the devices or substrate.

The compositions may comprise different types of MTMs within one category of MTMs, as defined herein, or the compositions may comprise different types of MTMs within two or more different categories of MTMs, as defined herein. Thus, the compositions of the invention include “cocktails” of different types of MTMs, wherein the composition can include 1, 2, 3, 4, 5, 6, 7, 8, 9, 10 or more different types of MTMs within a single composition.

The compositions of the invention may comprise mixtures of naturally-occurring MTMs (e.g. MBLs), mixture of both naturally-occurring MTMs (e.g. MBLs) and the engineered MTMs defined herein (e.g. FcMBLs), or mixtures of only engineered MTMs (e.g., FcMBLs).

Depending on the manner in which the MTMs are used, the compositions comprising one or more different type of MTM may include suitable carriers and diluents. Suitable carriers and diluents are commonly known and will vary depending on the MTM being used and the mode of use. Examples of suitable carriers and diluents include water, buffered water, saline, buffered saline, dextrose, glycerol, ethanol, and combinations thereof, propylene glycol, polysorbate 80 (Tween-80™), poly(ethylene)glycol 300 and 400 (PEG 300 and 400), hydrophilic and hydrophobic carriers, and combinations thereof. Hydrophobic carriers include, for example, fat emulsions, lipids, PEGylated phospholipids, polymer matrices, biocompatible polymers, lipospheres, vesicles, particles, and liposomes, other stabilizing agents, solubilizing agents and surfactants, buffers and preservatives, tonicity agents, bulking agents, and lubricating agents

The compositions of the invention may also comprise one or more antimicrobial agents. An MTM binds to and isolates one or more microbes or microbe components. An antimicrobial agent can optionally be included to treat (e.g. kill or inactivate) one or more known or suspected pathogens.

When the compositions comprise one or more antimicrobial agents, suitable agents include, but are not limited to, antibiotics, antivirals and antifungals. Antibiotics can be from classes including but not limited to Cephalosporin, Glycopeptide, Cyclic lipopeptide, Aminoglycoside, Macrolide, Oxazolidinone, Fluoroquinolones, Lincosamides, or Carbapenem. Antifungals can be from classes including but not limited to Polyenes, Azoles, Nucleoside Analog, Echinocandin, or Allylamine. Antivirals can be from classes including but not limited to CCR5 anatonists, Fusion inhibitors, Nucleoside/Nucleotide reverse transcriptase inhibitors (NRTIs), Non-nucleoside reverse transcriptase inhibitors (NNRTIs), Nucleotide reverse transcriptase inhibitors (NtRTIs), Integrase inhibitors, Protease inhibitors, DNA polymerase inhibitors, Guanosine analogs, Interferon-alpha, M2 ion channel blockers, Nucleoside inhibitors, NS5A polymerase inhibitors, NS3/4A protease inhibitors, Neuraminidase inhibitors, Nucleoside analogs, and Direct acting antivirals (DAAs). Examples of antimicrobials include but are not limited to aminoglycosides, ansamycins, beta-lactams, bis-biguanides, carbacephems, carbapenems, cationic polypeptides, cephalosporins, fluoroquinolones, glycopeptides, iron-sequestering glycoproteins, linosamides, lipopeptides, macrolides, monobactams, nitrofurans, oxazolidinones, penicillins, polypeptides, quaternary ammonium compounds, quinolones, silver compounds, sulfonamides, tetracyclines, and any combinations thereof.

Some exemplary antibiotics that may be included in the compositions of the invention include, but are not limited to, broad penicillins, amoxicillin (e.g., Ampicillin, Bacampicillin, Carbenicillin Indanyl, Mezlocillin, Piperacillin, Ticarcillin), Penicillins and Beta Lactamase Inhibitors (e.g., Amoxicillin-Clavulanic Acid, Ampicillin-Sulbactam, Benzylpenicillin, Cloxacillin, Dicloxacillin, Methicillin, Oxacillin, Penicillin G, Penicillin V, Piperacillin Tazobactam, Ticarcillin Clavulanic Acid, Nafcillin), Cephalosporins (e.g., Cephalosporin I Generation, Cefadroxil, Cefazolin, Cephalexin, Cephalothin, Cephapirin, Cephradine), Cephalosporin II Generation (e.g., Cefaclor, Cefamandole, Cefonicid, Cefotetan, Cefoxitin, Cefprozil, Cefmetazole, Cefuroxime, Loracarbef), Cephalosporin III Generation (e.g., Cefdinir, Ceftibuten, Cefoperazone, Cefixime, Cefotaxime, Cefpodoxime proxetil, Ceftazidime, Ceftizoxime, Ceftriaxone), Cephalosporin IV Generation (e.g., Cefepime), Macrolides and Lincosamides (e.g., Azithromycin, Clarithromycin, Clindamycin, Dirithromycin, Erythromycin, Lincomycin, Troleandomycin), Quinolones and Fluoroquinolones (e.g., Cinoxacin, Ciprofloxacin, Enoxacin, Gatifloxacin, Grepafloxacin, Levofloxacin, Lomefloxacin, Moxifloxacin, Nalidixic acid, Norfloxacin, Ofloxacin, Sparfloxacin, Trovafloxacin, Oxolinic acid, Gemifloxacin, Perfloxacin), Carbapenems (e.g., Imipenem-Cilastatin, Meropenem), Monobactams (e.g., Aztreonam), Aminoglycosides (e.g., Amikacin, Gentamicin, Kanamycin, Neomycin, Netilmicin, Streptomycin, Tobramycin, Paromomycin), Glycopeptides (e.g., Teicoplanin, Vancomycin), Tetracyclines (e.g., Demeclocycline, Doxycycline, Methacycline, Minocycline, Oxytetracycline, Tetracycline, Chlortetracycline), Sulfonamides (e.g., Mafenide, Silver Sulfadiazine, Sulfacetamide, Sulfadiazine, Sulfamethoxazole, Sulfasalazine, Sulfisoxazole, Trimethoprim-Sulfamethoxazole, Sulfamethizole), Rifampin (e.g., Rifabutin, Rifampin, Rifapentine), Oxazolidinones (e.g., Linezolid, Streptogramins, Quinupristin Dalfopristin), Bacitracin, Chloramphenicol, Fosfomycin, Isoniazid, Methenamine, Metronidazole, Mupirocin, Nitrofurantoin, Nitrofurazone, Novobiocin, Polymyxin, Spectinomycin, Trimethoprim, Colistin, Cycloserine, Capreomycin, Ethionamide, Pyrazinamide, Para-aminosalicylic acid, Erythromycin ethylsuccinate, and the like.

Some exemplary antifungals that may be included in the compositions of the invention include, but are not limited to, polyene antifungals, Amphotericin B, Candicidin, Filipin, Hamycin, Natamycin, Nystatin, Rimocidin, imidazole antifungals, triazole antifungals, thiazole antifungals, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Triazoles[edit], Albaconazole, Efinaconazole, Epoxiconazole, Fluconazole, Isavuconazole, Itraconazole, Posaconazole, Propiconazole, Ravuconazole, Terconazole, Voriconazole, Abafungin, Allylamines, amorolfin, butenafine, naftifine, terbinafine, Echinocandins, Anidulafungin, Caspofungin, Micafungin, Aurones, Benzoic acid, Ciclopirox, Flucytosine, 5-fluorocytosin, Griseofulvin, Haloprogin, Tolnaftate, Undecylenic acid, Triacetin, Crystal violet, Castellani's paint, Orotomide, Miltefosine, Potassium iodide, Coal tar, Copper(II) sulfate, Selenium disulfide, Sodium thiosulfate, Piroctone olamine, Iodoquinol, clioquinol, Acrisorcin, Zinc pyrithione, and Sulfur. Additional antifungals known in the art can also be used.

Some exemplary antivirals that may be included in the compositions of the invention include, but are not limited to, Abacavir, Acyclovir, Adefovir, Amantadine, Ampligen, Amprenavir, antiretroviral, Arbidol, Atazanavir, Atripla, Boceprevir, Cidofovir, Combivir, Daclatasvir, Darunavir, Delavirdine, Dasabuvir, Didanosine, Docosanol, Dolutegravir, Doravirine, Ecoliever, Edoxudine, Efavirenz, Elbasvir, Emtricitabine, Enfuvirtide, Entecavir, Etravirine, Famciclovir, Fomivirsen, Fosamprenavir, Foscarnet, Fosfonet, Fusion inhibitor, Ganciclovir, Gemcitabine, Glecaprevir, Grazoprevir, Ibacitabine, Idoxuridine, Imiquimod, Imunovir, Indinavir, Inosine, Integrase inhibitor, Interferon, Interferon type I, Interferon type II, Interferon type III, Lamivudine, Ledipasvir, Lopinavir, Lopiravir, Loviride, Maraviroc, Methisazone, Moroxydine, Nelfinavir, Nevirapine, Nexavir, Nitazoxanide, Norvir, Nucleoside analogues, Ombitasvir, Oseltamivir (Tamiflu), Paritaprevir, Peglyated Interferon-alpha, Peginterferon alfa-2a, Penciclovir, Peramivir, Pibrentasvir, Pleconaril, Podophyllotoxin, Protease inhibitor, Pyramidine, Raltegravir, Reverse transcriptase inhibitor, Ribavirin, Rilpivirine, Rimantadine, Ritonavir, Saquinavir, Simeprevir, Sofosbuvir, Stavudine, Synergistic enhancer (antiretroviral), Telaprevir, Telbivudine, Tenofovir, Tenofovir disoproxil, Tipranavir, Trifluridine, Trizivir, Tromantadine, Truvada, Valaciclovir (Valtrex), Valganciclovir, Velpatasvir, Vicriviroc, Vidarabine, Viramidine, Voxilaprevir, Zalcitabine, Zanamivir (Relenza), Zidovudine. Additional antivirals known in the art can also be used.

The compositions of the invention can take many different forms, varying widely based on (i) the identity of the MTMs in the composition, (ii) the identity of other components in the composition, and (iii) the intended use of the composition, to name only a few of the relevant factors.

In some aspects, MTM compositions (e.g., engineered MTMs as described therein), methods, systems, and assays are further described in at least one of the following: U.S. provisional application numbers 61/296,222, 61/508,957, 61/604,878, 61/605,052, 61/605,081, 61/788,570, 61/846,438, 61/866,843, 61/917,705, 62/201,745, 62/336,940, 62/543, 614; PCT application numbers PCT/US2011/021603, PCT/US2012/047201, PCT/US2013/028409 , PCT/US2014/028683, PCT/US2014/046716, PCT/US2014/071293, PCT/U52016/045509, PCT/US2017/032928; U.S. patent application Ser. Nos. 13/574,191, 14/233,553, 14/382,043, 14/766,575, 14/831,480, 14/904,583, 15/105,298, 15/415,352, 15/483,216, 15/668,794, 15/750,788, 15/839,352, 16/059,799, 16/302,023, 16/553,635; and U.S. Pat. Nos. 9,150,631, 9,593,160, 9,632,085, 9,791,440, and 10,435,457; the contents of each of which are incorporated by reference herein in their entireties.

### Labels

The MTMs of the present invention may be labeled to allow them to be detected after binding to microbes or microbial components. The identity of the detectable label is limited only in that it can be discerned by the human eye or via a detector in the context of the detection device. Suitable detectable labels include colored or fluorescent particles, such a Europium particles or colloidal gold. Other acceptable labels include latex, which may itself be tagged with colored or fluorescent dyes, and magnetic or paramagnetic components. A further detectable label is a plasmonic fluor, wherein instead of assaying for a color change, one detects fluorescence. Ultrabright fluorescent nanolabels can also be used to improve the limit of detection in the detection devices of the invention, compared with conventional fluorophores.

Other detectable labels include, but are not limited to, an enzyme (e.g., peroxidase, alkaline phosphatase, glucose oxidase), a metal (e.g., gold for electron microscopy applications), a fluorescent marker (e.g., for immunofluorescence and flow cytometry applications, including CYE dyes, fluorescein isothiocyanate, rhodamine, phycoerytherin, phycocyanin, allophycocyanin, o-phthaldehyde and fluorescamine), a fluorescence-emitting metals (e.g., ¹⁵²Eu), a radioactive marker (e.g., radioisotopes for diagnostic purposes, including ³H, ¹³¹I, ³⁵S, ¹⁴C, and ¹²⁵I), a chemiluminescent marker (e.g., luminol, luciferin, isoluminol, theromatic acridinium ester, imidazole, acridinium salt and oxalate ester), and a protein tag (e.g., biotin, phycobiliprotein, c-Myc, HA, VSV-G, HSV, FLAG, V5, or HIS).

### Side Groups

The MTMs of the invention can be engineered to display side groups that augment, enhance or otherwise alter selected characteristics of the MTMs. For example, the MTMs of the invention may be engineered to display polyfluoro groups on any portion of the molecule. Such groups include fluoropolymers comprising terminal polyfluoro-oligomeric groups. These groups can aid in reducing thrombosis that may result, for example, when blood comes into contact with non-self surfaces. Coating of such surfaces with MTMs displaying polyfluoro-groups can reduce coagulation.

### Devices

The present invention includes devices comprising MTMs (and related methods) to detect, capture and/or identify microbes and microbial components in a sample or in the environment. The devices may also be used in the treatment and prevention of microbial infections in a subject. These devices generally comprise a substrate onto which a composition comprising MTMs of the invention is applied.

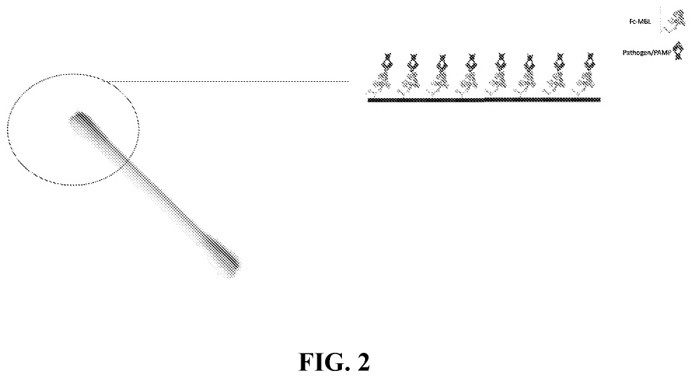

In some aspects, a composition comprising MTMs of the present invention is coated on a device comprising a substrate. In some aspects, the device comprise a collection device such as a swab configured to collect a sample, as shown in FIG. 2. Examples of a swab include but are not limited to a nasal, oral or ocular swab, any substrate commonly utilized in biological sample collection or environmental sample collection. Any or all of the portion of the swab that contacts a sample can be coated with a composition comprising MTMs of the present invention. The substrate can comprise any suitable material including but not limited to as plastic, cloth, fiber, fabric, such as cotton, metal such as a magnetic bead, a porous paper, and any combination thereof.

In some aspects, the device comprises a garment, e.g. a face covering such as a mask or face shield, or any type of outerwear such as a shirt, pants, a hat, and the like, all as are known in the art, coated with a composition comprising MTMs of the invention. Examples of masks include but are not limited to a surgical mask, a KN95 mask, an N95 mask, or a cloth or fabric mask. Any portion or all of the garment can be coated with a composition comprising MTMs. The substrate can comprise any suitable material including but not limited to as plastic, cloth, fiber, fabric, gel, and any combination thereof.

In some aspects, the device comprises a bandage, such as a cloth or paper bandage, a liquid bandage, a hydrogel bandage, a foam bandage, or an adhesive bandage, as are known in the art, coated with a composition comprising MTMs of the invention. Any portion or all of the bandage can be coated with a composition comprising MTMs.

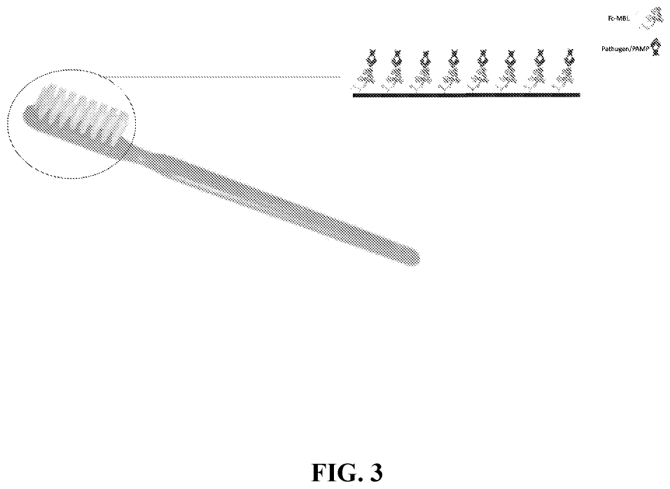

In some aspects, the device comprises a toothbrush as shown in FIG. 3, where the bristles are coated with a composition comprising MTMs of the present invention. In some aspects, the toothbrush further comprises an assay such as a lateral flow assay to monitor and detect pathogen materials as well as the host response in the oral cavity in real time. A non-limiting example of a lateral flow assay is described in PCT/US21/37021, filed on Jun. 11, 2021, which is incorporated by reference in its entirety herein. In some aspects, the device further comprises a communication module, to wirelessly or through wired communication, communicate with a database, e.g., a cloud, to store personal healthcare information, and in some cases, for broader healthcare monitoring, e.g., during a pandemic, or to monitor the onset of a pandemic.

In some aspects, the device can be configured to be applied to a subject at a clinician's office. In some aspects, the device can be configured to be used in the home.

The device can be applied to a subject preventatively to prevent one or more pathogens from entering a subject, e.g., into the eye(s), nose, mouth, and/or respiratory system including the airway, lungs and blood vessels, and blood. The device comprises a composition comprising MTMs where the MTMs act by binding to one or more microbes or microbe components and preventing the one or more microbe or microbe components, thus immobilizing it and preventing it from entering the eye(s), nose, mouth, and/or respiratory system including the airway, lungs and blood vessels, and ultimately the blood. These types of coated substrates may be particularly advantageous in preventing a respiratory-transmitted infection such as SARS-CoV-2. For example, health authorities recommend that masks be worn by ill individuals to prevent outward transmission. Pathogens such as the SARS-CoV-2 virus spread between humans through direct or indirect contact and respiratory droplets (including larger droplets that fall rapidly near the source as well as coarse aerosols with an aerodynamic diameter larger than 5 microns) and fine-particle aerosols (droplets and droplet nuclei with an aerodynamic diameter of less than or equal to 5 microns). Some medical grade masks, such as N95 and KN95 masks, are configured to filter the virus, but may not be widely available to the public. Therefore, any mask coated with a composition comprising MTMs of the present invention is useful to prevent or reduce the transmission of a pathogen.

Any of the coated substrates described herein can be applied to a subject after exposure to a pathogen, for example, to decrease the pathogen load on the subject and/or to treat a subject having one or more infections. In some aspects, if a subject is exposed to a known pathogen, a particular antimicrobial, based on the pathogen, can be selected and added to the composition and then coated on a substrate.

There are various methods for coating a substrate with a composition of the present invention. Immobilization (via coating) of MTMs onto a surface can be either non-specific (e.g., by adsorption to the surface) or specific (e.g. where another molecule, such as a linker, immobilized on the surface is used to capture the MTM). MTMs may be linked to the surface through one or more linkers which may be cleavable to accommodate release or elution of the bound target molecules for subsequent analysis. Coating also includes, but is not limited to, pre-treatment of a substrate using plasma treatment, corona treatment, or flame treatment.

An exemplary plasma treatment includes drying the substrate to eliminate residual moisture with filtered, compressed air; treating the substrate with a carbon dioxide plasma to introduce carboxylate moieties to enable the chemical crosslinking of the one or more MTMs of the composition. The substrate is contacted with a buffer, for example, 2-(N-morpholino) ethanesulfonic acid (MES) buffer containing FcMBL and 1-ethyl-3-(3-dimethylaminopropyl) carbodiimide (EDC), a zero-length carboxyl-to-amino crosslinker. Then, the substrate is incubated overnight at 2-8 degrees Celsius during which time the EDC crosslinks the FcMBL protein to the inner lumen of the hollow fibers. Subsequently, for example, approximately 24 hours later, the FcMBL-coupled devices are washed with excess phosphate buffered saline (PBS) solution containing 10 mM ethylenediaminetetraacetic acid (EDTA) (PBS/EDTA) through both the blood and dialysate compartments to remove the buffer, unbound protein, and EDC.

A coronoa treatment is a surface modification technique that uses a low temperature corona discharge plasma to impart changes in the properties of a surface. An exemplary method includes generating a corona plasma by the application of high voltage to an electrode that has a sharp tip, where the plasma forms at the tip. A linear array of electrodes can be used to create a curtain of corona plasma. The substrate may be passed through the corona plasma curtain in order to change the surface energy of the material, and then a composition comprising MTMs can be applied to the surface. In some aspects, a non-covalent bonding process can be used.

A flame treatment comprises applying a gas flame to the surface of a material to improve adhesion. By rapidly applying intense heat to a surface, molecular chains are broken, and polar functional groups are added. Flame treatment also burns off dust, fibers, oils, and other surface contaminants. Following such treatment, a composition comprising MTMs can be applied to the surface.

### Methods

The devices of the invention may be used in a wide variety of applications including, but not limited to, methods of detecting the presence of a microbe or microbial component in a bodily fluid of a subject. Such methods include contacting a bodily fluid of the subject with a device of the invention under conditions that permit binding of microbes or microbial components by MTMs displayed by the device, thus detecting microbes or microbial components in the bodily fluid of the subject. In one aspect, the microbe is a bacteria. In another aspect, the microbe is a virus. In further aspect, the microbe is a fungus. In further aspect, the microbe is a protozoan. Optionally, such methods can include one or more of the following additional steps: (i) quantifying the amount of microbe or microbial component in the bodily fluid; (ii) identifying the microbe in the bodily fluid. When the MTM used in conjunction with the device is a species-specific MTM, for example, the microbe being detected by the device can be identified to the taxonomic level of species. However, when the MTM(s) used in conjunction with the device are not species-specific, i.e. the MTM(s) recognize and bind a family or genus of microbes and cannot identify the microbe to the taxonomic level of species, a further identification means may be used to identify the microbe to the selected taxonomic level.

The present invention includes methods for treating infectious disease using the devices of the invention. Such methods comprise applying a device or substrate comprising a composition comprising MTMs to a subject (e.g., a human or an animal subject) in need thereof. In some aspects, applying a device or substrate to a subject can include preventing one or more pathogens from entering a subject, e.g., into the eye(s), nose, mouth, and/or respiratory system, including the airway, lungs and blood vessels, and blood, for example, where the device is a garment worn by the subject. In some aspects, applying a device or substrate to a subject can include capturing/collecting a sample, including one or more microbes or microbe components, from a subject. As an example, a device can comprise a collection device such as a cotton swab, where the swab can be inserted into the front portion of the nose or a deeper portion of the nose such as the pharyngeal or the oropharyngeal sputum. Subsequently, the swab comprising the sample can be placed in a collection media. In some aspects, the device comprising the sample can be placed in a collection media such as a transfer buffer, for example, when the collection device comprises a cotton swab used in a “at home” test kit, and returned to a lab where a lab performs subsequent sample preparation and analysis, e.g., using qPCR to identify a microbe or microbe components in the sample.

In some aspects, applying a device or substrate to a subject can include capturing/collecting a sample, including one or more microbes or microbe components, from a dialysis device to detect/identify MAMPs in the dialysis fluid. As an example, a device can comprise a collection device such as a cotton swab, where the swab can be inserted into the dialysis fluid. Subsequently, the swab comprising the sample can be placed in a collection media. In some aspects, the device comprising the sample can be placed in a collection media and prepared for subsequent sample preparation and analysis, e.g., using qPCR to identify MAMPs in the sample.

In any of these aspects, the method can further comprise preparing a sample for analysis, e.g., isolating the microbe or microbe components which can include eluting the microbe or microbe components from the device. In some aspects, the step of isolating comprises washing the support with a buffer to remove unbound cells or biomolecules. The buffer can be any buffer as described herein, including but not limited to tris-buffered-saline, phosphate buffer saline, water, HPLC grade H₂O, comprising octyl-β-D-glucopyranoside and/or calcium (TB SG Ca²⁺) with surfactants. In some aspects, the step of washing can be performed at least 1, at least 2, at least 3, at least 4, or at least 5 times.

In some aspects, calcium depletion may be used to elute microbes or microbe components from the device. In some aspects, the sample is place in Trazol for nucleic acid enrichment.

The microbes and/or microbial matter (e.g., MAMPs) bound to MTM-coated (e.g., lectin-coated) solid substrates (e.g., mask, garment, collection swab or a toothbrush) or a solid surface can be detected by any methods known in the art or as described herein. Examples of detection methods can include, but are not limited to, spectrometry, electrochemical detection, polynucleotide detection, fluorescence anisotropy, fluorescence resonance energy transfer, electron transfer, enzyme assay, magnetism, electrical conductivity, isoelectric focusing, chromatography, immunoprecipitation, immunoseparation, aptamer binding, filtration, electrophoresis, use of a CCD camera, immunoassay, ELISA, Gram staining, immunostaining, microscopy, immunofluorescence, western blot, polymerase chain reaction (PCR), RT-PCR, isothermal amplification, fluorescence in situ hybridization, sequencing, mass spectrometry, raman spectrometry, surface plasmon resonance, or substantially any combination thereof. The captured microbe can remain bound on the PRR-coated solid substrates during detection and/or analysis, or be isolated form the PRR-coated solid substrates prior to detection and/or analysis.

In some embodiments of any of the aspects, the microbes and/or microbial matter (e.g., MAMPs) bound to PRR-coated (e.g., lectin-coated) solid substrates (e.g., polymeric or magnetic particles or beads) can be detected by ELLecSA as defined herein, an example which is described in detail in the section “An exemplary enzyme-linked lectin sorbent assay (ELLecSA)” below. Additional information various embodiments of FcMBL based assays can be found, e.g., in PCT application no. PCT/US2012/047201 and no. PCT/US2013/028409, the contents of all of which are incorporated herein by reference in their entireties.

The invention is also directed to methods of detecting a microbe in a sample, comprising contacting a sample suspected of containing a microbe with a device or substrate of the invention comprising MTMs under conditions permitting binding of a microbe or a component of a microbe by MTMs coated on, or otherwise displayed by, the device or substrate, thereby detecting a microbe in a sample.

The invention is also directed to methods of detecting a microbial infection in a subject, comprising contacting a biological sample of a subject suspected of having a microbial infection with a device or substrate of the invention comprising MTMs under conditions permitting binding of a microbe or a component of a microbe by MTMs coated on, or otherwise displayed by, the device or substrate, thereby detecting a microbial infection in the subject.

The invention is also directed to methods of diagnosing a microbial infection in a subject, comprising contacting a biological sample of a subject suspected of having a microbial infection with a device or substrate of the invention comprising MTMs under conditions permitting binding of a microbe or a component of a microbe by MTMs coated on, or otherwise displayed by, the device or substrate, thereby diagnosing a microbial infection in the subject.

The invention is also directed to methods of treating a microbial infection in a subject, comprising contacting a bodily fluid of a subject having a microbial infection with a device or substrate of the invention comprising MTMs under conditions permitting binding of a microbe or a component of a microbe by MTMs coated on, or otherwise displayed by, the device or substrate, thereby treating a microbial infection in the subject.

The invention is also directed to methods of filtering a microbe or microbial component from a fluid, comprising contacting a fluid containing a microbe or microbial component with a device or substrate of the invention comprising MTMs under conditions permitting binding of a microbe or a component of a microbe by MTMs coated on, or otherwise displayed by, the device or substrate, thereby filtering a microbe or microbial component from a fluid.

The invention is also directed to methods of inhibiting or preventing entry of a microbe or microbial component into a subject, comprising applying a device or substrate comprising MTMs of the invention to a subject. In certain aspects, the device is a mask covering the mouth and nose of the subject. In certain other aspects, the device is a bandage covering a wound of the subject.

In relevant aspects of these methods, the methods may further comprise identifying the microbe bound by the MTMs.

In relevant aspects of these methods, the sample may be a biological sample. For example, the biological sample may be blood.

In relevant aspects of these methods, the device may comprise two or more MTMs having different binding specificities.

In relevant aspects of these methods, the device may comprise one or more MTMs set forth in SEQ ID NOs: 6, 7, 8 and 9.

In some embodiments of any of the aspects, compositions (e.g., engineered microbe targeting molecules as described further therein), methods, systems, and assays are further described in at least one of the following: U.S. Provisional Applications 61/296,222, 61/508,957, 61/604,878 , 61/605,052 , 61/605,081, 61/788,570, 61/846,438, 61/866,843, 61/917,705, 62/201,745, 62/336,940, 62/543, 614; PCT application numbers PCT/US2011/021603, PCT/US2012/047201, PCT/US2013/028409 , PCT/US2014/028683, PCT/US2014/046716, PCT/US2014/071293, PCT/US2016/045509, PCT/US2017/032928; U.S. patent application Ser. Nos. 13/574, 191, 14/233,553, 14/382,043 , 14/766,575, 14/831,480, 14/904,583, 15/415,352, 15/483,216 , 15/668,794, 15/750,788, 15/839,352, 16/059,799, 16/302,023, 16/553,635; and U.S. Pat. Nos. 9,150,631, 9,593,160, 9,632,085, 9,791,440, and 10,435,457; the contents of each of which are incorporated by reference herein in their entireties.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further aspects thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various aspects and equivalents thereof.

What is claimed is:

1. A device comprising at least one substrate coated with, or otherwise displaying, one or more microbe-targeting molecules (MTMs).

2. The device of claim 1, wherein the substrate is coated with a composition comprising one or more collectin-based MTMs, wherein the MTMs comprise at least one collectin microbe-binding domain and at least one additional domain,
wherein the collectin microbe-binding domain comprises the carbohydrate recognition domain (CRD) of a collectin selected from the group consisting of
(i) mannose-binding lectin (MBL),
(ii) surfactant protein A (SP-A),
(iii) surfactant protein D (SP-D),
(iv) collectin liver 1 (CL-L1),
(v) collectin placenta 1 (CL-P1),
(vi) conglutinin collectin of 43 kDa (CL-43),
(vii) collectin of 46 kDa (CL-46),
(viii) collectin kidney 1 (CL-K1),
(ix) conglutinin, and
(x) a sequence variant having at least 85% sequence identity to any one of (i)-(ix), and
wherein the at least one additional domain is one or more domains selected from the group consisting of
(xi) a collectin cysteine-rich domain,
(xii) a collectin collagen-like domain,
(xiii) a collectin coiled-coil neck domain,
(xiv) a ficolin short N-terminal domain,
(xv) a ficolin collagen-like domain,
(xvi) a TLR transmembrane helix,
(xvii) a TLR C-terminal cytoplasmic signaling domain,
(xviii) an oligomerization domain,
(xix) a signal domain,
(xx) an anchor domain,
(xxi) a collagen-like domain,
(xxii) a fibrinogen-like domain,
(xxiii) an immunoglobulin domain,
(xxiv) an immunoglobulin-like domain, and
(xxv) a sequence variant having at least 85% sequence identity to any one of (xi)-(xxiv).

3. The device of claim 2, wherein the collectin microbe-binding domain comprises the CRD of MBL or a sequence variant thereof having at least 85% sequence identity to the CRD of MBL.

4. The device of claim 2 or 3, wherein the CRD of MBL comprises the amino acid sequence of any one of SEQ ID NOs:1, 2, 3, 4, and 5 or a sequence variant thereof having at least 85% sequence identity to any one of SEQ ID NOs:1, 2, 3, 4, and 5.

5. The device of claim 2, wherein the at least one additional domain is an immunoglobulin domain.

6. The device of claim 2 or 5, wherein the immunoglobulin domain comprises the amino acid sequence of SEQ ID NO:12 or a sequence variant thereof having at least 85% sequence identity to SEQ ID NO:12.

7. The device of claim 2 or 5, wherein the MTM is one or more of SEQ ID NOs: 6-9, or a sequence variant thereof having at least 85% sequence identity to any one of SEQ ID NOs:6-9.

8. The device of claim 1, wherein the substrate is coated with a composition comprising one or more ficolin-based MTMs, wherein the MTMs comprise at least one ficolin microbe-binding domain and at least one additional domain,
wherein the ficolin microbe-binding domain comprises the fibrinogen-like domain of a ficolin selected from the group consisting of
(i) ficolin 1,
(ii) ficolin 2,
(iii) ficolin 3, and
(iv) a sequence variant having at least 85% sequence identity to any one of (i)-(iii), and
wherein the at least one additional domain is one or more domains selected from the group consisting of
(v) a ficolin short N-terminal domain,
(vi) a ficolin collagen-like domain,
(vii) a collectin cysteine-rich domain,
(viii) a collectin collagen-like domain,
(ix) a collectin coiled-coil neck domain,
(x) a TLR transmembrane helix,
(xi) a TLR C-terminal cytoplasmic signaling domain,
(xii) an oligomerization domain,
(xiii) a signal domain,
(xiv) an anchor domain,
(xv) a collagen-like domain,
(xvi) a fibrinogen-like domain,
(xvii) an immunoglobulin domain,
(xviii) an immunoglobulin-like domain, and
(xix) a sequence variant having at least 85% sequence identity to any one of (v)-(xviii).

9. The device of claim 8, wherein the ficolin microbe-binding domain comprises the fibrinogen-like domain of any one of SEQ ID NOs:21, 22 and 23, or a sequence variant thereof having at least 85% sequence identity to any one of SEQ ID NOs:21, 22 and 23.

10. The device of claim 8 or 9, wherein the at least one additional domain is an immunoglobulin domain.

11. The device of any one of claims 8-10, wherein the immunoglobulin domain comprises the amino acid sequence of SEQ ID NO:12 or a sequence variant thereof having at least 85% sequence identity to SEQ ID NO:12.

12. The device of claim 1, wherein the substrate is coated with a composition comprising one or more toll-like receptor (TLR)-based MTMs, wherein the MTMs comprise at least one TLR microbe-binding domain and at least one additional domain,
wherein the TLR microbe-binding domain comprises the N-terminal ligand-binding domain of a TLR selected from the group consisting of
(i) TLR1,
(ii) TLR2,
(iii) TLR3,
(iv) TLR4,
(v) TLR5,
(vi) TLR6,
(vii) TLR7,
(vii) TLR8,
(viii) TLR9,
(ix) TLR10, and
(x) a sequence variant having at least 85% sequence identity to any one of (i)-(ix), and
wherein the at least one additional domain is one or more domains selected from the group consisting of
(xi) a TLR transmembrane helix,
(xii) a TLR C-terminal cytoplasmic signaling domain,
(xiii) a ficolin short N-terminal domain,
(xiv) a ficolin collagen-like domain,
(xv) a collectin cysteine-rich domain,
(xvi) a collectin collagen-like domain,
(xvii) a collectin coiled-coil neck domain,
(xviii) an oligomerization domain,
(xix) a signal domain,
(xx) an anchor domain,
(xxi) a collagen-like domain,
(xxii) a fibrinogen-like domain,
(xxiii) an immunoglobulin domain,
(xxiv) an immunoglobulin-like domain, and
(xxv) a sequence variant having at least 85% sequence identity to any one of (xi)-(xxiv).

13. The device of claim 12, wherein the TLR microbe-binding domain comprises the N-terminal ligand-binding domain of any one of SEQ ID NOs:24-33 or a sequence variant thereof having at least 85% sequence identity to any one of SEQ ID NOs:24-33.

14. The device of claim 12 or 13, wherein the at least one additional domain is an immunoglobulin domain.

15. The device of any one of claims 12-14, wherein the immunoglobulin domain comprises the amino acid sequence of SEQ ID NO:12 or a sequence variant thereof having at least 85% sequence identity to SEQ ID NO:12.

16. The device of claim 1, wherein the substrate is coated with a composition comprising one or more MTMs, wherein the one or more MTMs is selected from any of the MTMs of claims 2-15.

17. The device of claim 16, further comprising at least one naturally-occurring MTM.

18. The device of claim 16, further comprising one or more antimicrobial agents.

19. The device of claim 17, further comprising one or more antimicrobial agents.

20. The device of claim 16, comprising at least two collectin-based MTMs of claim 2, wherein at least one of the collectin-based MTMs is an FcMBL of SEQ ID NO:6, 7, 8 or 9 or a sequence variant thereof having at least 85% sequence identity to SEQ ID NO:6, 7, 8 or 9.

21. The device according to any preceding claim, wherein the substrate is selected from the group consisting of plastic, cloth, fiber, fabric, metal, porous paper, and any combination thereof.

22. The device of claim 21, wherein the cloth or fiber is cotton.

23. The device of claim 21, wherein the metal is a magnetic bead.

24. The device according to any preceding claim, wherein the device is a bandage or a garment selected from the group consisting of a face covering, a shirt, a pant, and a hat.

25. The device of claim 24, wherein the face covering is a surgical mask, a KN 95 mask, an N95 mask, or a cloth or fabric mask.

26. The device of claim 24, wherein the bandage is a cloth or paper bandage, a liquid bandage, a hydrogel bandage, a foam bandage, or an adhesive bandage.

27. The device according to any preceding claim, wherein the device is a cotton swab or a toothbrush.

28. A method of detecting a microbe in a sample, comprising contacting a sample suspected of containing a microbe with a device of claim 1 under conditions permitting binding of a microbe or a component of a microbe by MTMs coated on, or otherwise displayed by, the substrate of the device, thereby detecting a microbe in a sample.

29. A method of detecting a microbial infection in a subject, comprising contacting a biological sample of a subject suspected of having a microbial infection with a device of claim 1 under conditions permitting binding of a microbe or a component of a microbe by MTMs coated on, or otherwise displayed by, the substrate of the device, thereby detecting a microbial infection in the subject.

30. A method of diagnosing a microbial infection in a subject, comprising contacting a biological sample of a subject suspected of having a microbial infection with a device of claim 1 under conditions permitting binding of a microbe or a component of a microbe by MTMs coated on, or otherwise displayed by, the substrate of the device, thereby diagnosing a microbial infection in the subject.

31. A method of treating a microbial infection in a subject, comprising contacting a bodily fluid of a subject having a microbial infection with a device of claim 1 under conditions permitting binding of a microbe or a component of a microbe by MTMs coated on, or otherwise displayed by, the substrate of the device, thereby treating a microbial infection in the subject.

32. A method of filtering a microbe or microbial component from a fluid, comprising contacting a fluid containing a microbe or microbial component with a device of claim 1 under conditions permitting binding of a microbe or a component of a microbe by MTMs coated on, or otherwise displayed by, the substrate of the device, thereby filtering a microbe or microbial component from a fluid.

33. A method of inhibiting entry of a microbe or microbial component into a subject, comprising applying a device of claim 1 to a subject.

34. A method of preventing entry of a microbe or microbial component into a subject, comprising applying a device of claim 1 to a subject.

35. The method of claim 33 or 34, wherein the device is a mask covering the mouth and nose of the subject.

36. The method of claim 33 or 34, wherein the device is a bandage covering a wound of the subject.
